(19) United States
(12) Patent Application Publication
(10) Pub. No.: US 2026/0217790 A1
(43) Pub. Date: Jul. 30, 2026

(54) MODULATING THE PROPERTIES OF GPCR-BASED SENSORS VIA C-TERMINUS ISOFORMS

(71) Applicant: Georgia Tech Research Corporation, Atlanta, GA (US)

(72) Inventors: Pamela Peralta-Yahya, Atlanta, GA (US); Paola Lizeth Marquez-Gomez, Roswell, GA (US)

(21) Appl. No.: 19/458,360

(22) Filed: Jan. 23, 2026

Related U.S. Application Data
(60) Provisional application No. 63/749,351, filed on Jan. 24, 2025.

(51) Int. Cl. C07K 14/705; C07K 14/47; C12N 15/81; C12Q 1/02

(52) U.S. Cl. CPC C07K 14/70571; C07K 14/4702; C12N 15/81; C12Q 1/02

(57) ABSTRACT

G-protein coupled receptors (GPCRs) play a key role in chemical biosensing, detecting chemicals from odorants and hormones to neurotransmitters and peptides. GPCR-based sensors in yeast can be rapidly engineered by coupling human GPCRs to the yeast mating pathway, resulting in cell fluorescence or luminescence upon chemical detection. Modulating the properties of GPCR-based sensors including their dynamic and linear ranges is non-trivial, often requiring the engineering of the yeast cell machinery. Disclosed herein is the use of GPCR C-terminus isoforms and mutants to modulate the properties of chemical biosensors.

### CROSS-REFERENCE TO RELATED APPLICATION[S]

This application claims priority to, and the benefit of, U.S. Provisional Patent Application Ser. No. 63/749,351, entitled “MODULATING THE PROPERTIES OF GPCR-BASED SENSORS VIA C-TERMINUS ISOFORMS” and filed on Jan. 24, 2025, the entire contents of which are incorporated herein by reference as if set forth in its entirety.

### STATEMENT OF GOVERNMENT INTEREST

This invention was made with Government Support under Grant No. GM124871 awarded by the National Institutes of Health. The Government has certain rights in the invention.

### SEQUENCE LISTING

The instant application contains a Sequence Listing which has been submitted electronically in .XML format and is hereby incorporated by reference in its entirety. Said .XML copy, created on Jan. 24, 2026, is named “62021_1780_sequence_listing.xml” and is 38,787 bytes in size.

### BACKGROUND OF THE INVENTION

G-protein coupled receptors (GPCRs) detect chemical signals on the outside of the cell and transduce this information to the inside of the cell, resulting in the regulation of genomic targets. GPCRs thus play an important role in cell signaling, regulating physiological processes including neurotransmission, growth, energy metabolism, and cardiac function (Syrovatkina, et al. J Mol Biol 2016, 428:3850-68). The innate ability of GPCRs to bind a wide variety of chemicals has led to the development of GPCR-based sensors in yeast. Yeast GPCR-based sensors are constructed by expressing a human GPCR on the cell surface, linking its activation to the yeast mating pathway, ultimately leading to reporter gene expression. GPCR-based sensors have wide biotechnology uses, from serving as high-throughput screening platforms to accelerate drug discovery, to being embedded in point-of-care diagnostics, to metabolic engineering applications such as quantification of microbially produced metabolites (Patel, et al. Biochemistry 2023, 62:187-195).

Different GPCR-based sensor applications require different sensor properties, including dynamic and linear ranges. Although GPCR-based sensors can be quickly assembled —if the GPCR couples to the yeast machinery (Lengger, et al. ACS Sensors 2022, 7:1323-1335; Kapolka, et al. Proc Natl Acad Sci USA 2020, 117:13117-13126; Ehrenworth, et al. Biochemistry 2017, 56:5471-5475)—most sensors initially have limited dynamic ranges, which need to be optimized before use in the desired application.

### SUMMARY OF THE INVENTION

To date, the modulation of GPCR-based sensor properties has largely relied on swapping the yeast Ga-protein (GPA1) with yeast/mammalian Ga-protein chimeras (Lengger, et al. ACS Sensors 2022, 7:1323-1335; Kapolka, et al. Proc Natl Acad Sci USA 2020, 117:13117-13126). As disclosed herein, the GPCR C-terminus can be varied to achieve different sensor properties. Inspiration for this exploration is the fact that, in mammalian cells, the GPCR C-terminus alters receptor coupling to G-proteins, internalization, and membrane trafficking (Marti-Solano, et al. Nature 2020, 587:650-656). By focusing only on modifications to the GPCR C-terminus, the chemical binding site located at the N-terminus is left undisturbed.

GPCR C-terminus variations are present in nature. Fifty percent of human GPCRs undergo alternative mRNA splicing (Markovic, et al. Cell Mol Life Sci 2009, 66:3337-52), leading to GPCR isoforms with distinct tissue distributions (Marti-Solano, et al. Nature 2020, 587:650-656; Kilpatrick, et al. Trends Pharmacol Sci 1999, 20:294-301) and pharmacological responses (Pan, et al. Proc Natl Acad Sci USA 2009, 106:4917-22; Sato, et al. J Pharmacol Exp Ther 2005, 315:1354-61). For example, serotonin receptor 4 (5-HTR₄), a pharmacological target playing roles in conditions such as irritable bowel syndrome mood regulation and anxiety (Karayol, et al. Mol Psychiatr 2021, 26:2334-2349), has ten isoforms (Bockaert, et al. Cell Tissue Res 2006, 326:553-72) with a wide tissue distribution, from the brain and heart to the colon and testis (Coupar, et al. Curr Neuropharmacol 2007, 5:224-31; Medhurst, et al. Brain Res Mol Brain Res 2001, 90:125-34; Oladosu, et al. Mayo Clin Proc 2015, 90:1135-51; Cartier, et al. Eur J Endocrinol 2005, 153:939-47). To date, of the six 5-HTR families (5-HTR₁, 5-HTR₂, 5-HTR₄, 5-HTR₅, 5-HTR₆ and 5-HTR₇), yeast GPCR-based sensors for only two have been generated: 5-HTR₁ (5-HTR_{1A}, 5-HTR_{1B}, 5-HTR_{1D}, 5-HTR_{1E}) and 5-HTR₄ (5-HTR_{4B}) (Lengger, et al. ACS Sensors 2022, 7:1323-1335; Kapolka, et al. Proc Natl Acad Sci USA 2020, 117:13117-13126; Ehrenworth, et al. Biochemistry 2017, 56:5471-5475; Brown, et al. Yeast 2000, 16:11-22; Nakamura, et al. Biotechnol Bioeng 2015, 112:1906-15; Shaw, et al. Cell 2019, 177:782-796 e27; Bean, et al. Nat Commun 2022, 13:2882; Yasi, et al. ACS Synth Biol 2019, 8:2710-2717. The four 5-HTR₁ isoforms studied to date have sequence variations throughout the protein, including the N-terminus, i.e. near the orthosteric binding site, and intracellular loop 3.

G-protein coupled receptors (GPCRs) play a key role in chemical biosensing, detecting chemicals from odorants and hormones to neurotransmitters and peptides. GPCR-based sensors in yeast can be rapidly engineered by coupling human GPCRs to the yeast mating pathway, resulting in cell fluorescence or luminescence upon chemical detection. Modulating the properties of GPCR-based sensors including their dynamic and linear ranges is non-trivial, often requiring the engineering of the yeast cell machinery. Disclosed herein is the use of GPCR C-terminus isoforms and mutants to modulate the properties of chemical biosensors. As a proof-of-concept, nine naturally occurring serotonin receptor 4 (5-HTR₄)C-terminus isoforms were leveraged to construct serotonin sensors with dynamic ranges ranging from 2— to 8.5-fold increases in signal after activation, and linear ranges spanning 5 orders of magnitude, from 10⁻⁷-10⁻³ M serotonin. Interestingly, the 5-HTR₄ isoform-based sensors had different properties based on the chemical used to activate them, hinting at the potential differential activation of 5-HTR4 C-terminus isoforms in the body. Taken together, this work debuts the use of GPCR isoforms as a new strategy to rapidly modulate the dynamic and linear ranges of GPCR-based sensors in yeast.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A-1G2. Serotonin receptor 4 (5-HTR₄)C-terminus isoform-based sensors. FIG. 1A. Overview of the most common location of 5-HTR₄ C-terminus isoforms in the body. Information obtained from¹⁴⁻¹⁷. FIG. 1A was created in Biorender. Peralta-Yahya. (2024) https://BioRender.com/j17i302. FIG. 1B. Snake plots of the nine 5-HTR₄ C-terminus isoforms studied in this work. Snake plots were made using Protter²⁷. FIG. 1C. Sequences of the C-terminus of the nine 5-HTR₄ C-terminus isoforms. Green: S/T phosphorylation consensus for β-arrestin dependent endocytosis. Blue: L358 the last consensus amino acid. Red: Arg³⁵⁹. Grey: Ser³⁵⁹. Sequence alignment done using Clustal X. FIG. 1D. Schematic of 5-HTR₄ C-terminus isoform-based sensor in yeast. Human 5-HTR₄ isoform (blue) is expressed on the yeast cell surface. Upon serotonin binding, the receptor activates the yeast mating pathway (yellow) via the G_{β/γ} subunits ultimately resulting in the expression of luciferase (purple). FIG. 1E. mRNA levels for each of the 5-HTR₄ C-terminus isoforms when compared to the housekeeping gene actin (ACT1). The data was collected using the same cells used for FIG. 1F1-1F2. All experiments were performed in biological triplicates. The bars represent mean±standard error of mean (SEM), n=3. Data were analyzed using one-way ANOVA with Tukey's multiple comparison using GraphPad. The ΔCt values between the isoforms were not statistically different. FIGS. 1F1-1F2. Dose response curves of the nine single integrated 5-HTR₄ C-terminus isoform-based sensors in the presence of serotonin. D=DMSO, carrier solvent. Sensor strain genotype: W303 Δste2, Δste12, Δfar1, His3: P_{Adh1}-5-HTR4 C-terminus isoform, pRS415-P_{Fig1}-Nanoluc. FIGS. 1G1-1G2. Dose response curves of the nine double integrated 5-HTR₄ C-terminus isoform-based sensors in the presence of serotonin. D=DMSO, carrier solvent. Sensor strain genotype: W303 Δste2, Δste12, Δfar1, His3: P_{Adh1}-5-HTR4 C-terminus isoform, Trp1: P_{Adh1}-5-HTR4 C-terminus isoform, pRS415-P_{fig1}-Nanoluc. For 1F1-1F2 and 1G1-1G2, all experiments were performed in biological triplicates. The bars represent mean±standard error of mean (SEM), n=3, *p≤0.05. Data were analyzed using one-way ANOVA with Dunnett's multiple comparison between DMSO and serotonin treatments using GraphPad.

FIGS. 2A-2D. Ligand influence on 5-HTR₄ C-terminus isoform-based sensor properties. FIG. 2A. Heat map of the root mean square deviation (RMSD) of the active site of the AlphaFold models of the nine 5-HTR₄ C-terminus isoforms and the Cryo-EM structure of 5-HR_{4B} (PDB 7XT9). FIG. 2B. Structural alignment of the 5-HTR_{4B} Cryo-EM structure (PDB: 7XT9, purple), and AlphaFold models of 5-HTR_{4B} (cyan) and 5-HTR_{4G} (orange). FIG. 2C. Structural comparison of serotonin and the three ligands tested: tegaserod, which shares the 5-hydroxyindole moiety with serotonin and metoclopramide and cisapride which relies on a hydroxyaniline moiety. FIG. 2D. Fold increase in signal after activation of single integrated 5-HTR₄ C-terminus isoform-based sensor with serotonin(S), tegaserod (T), metoclopramide (M) and cisapride (C). All experiments were performed in biological triplicates. Raw data can be found in FIGS. 4A-4I and FIG. 5.

FIG. 3 shows AlphaFold structural predictions for 5-HTR₄ C-terminus isoform. Structures visualized using PyMOL. Structures in grey. C-terminus starting at L³⁵⁸ in cyan (denoted with arrows).

FIGS. 4A-4I. Dose response curves of 5-HTR4 C-terminus isoform-based sensors with serotonin, tegaserod, metoclopramide and cisapride. All experiments were performed in biological triplicates. The bars represent mean±standard error of mean (SEM), n=3, *p≤0.05. Data were analyzed using two-way ANOVA with Tukey's multiple comparison between signal in the presence of serotonin vs. signal in the presence of other compounds using GraphPad.

FIG. 5 Data on fold increase in signal after activation of single integrated 5-HTR₄ C-terminus isoform-based sensor with serotonin(S), tegaserod (T), metoclopramide (M) and cisapride (C).

FIG. 6 illustrates a GPCR-based chemical biosensor.

FIGS. 7A and 7B show embodiments of a GPCR-based chemical biosensor having an optional amplification unit that can directly (FIG. 7A) or indirectly (FIG. 7B) amplify a signal generated by a sensing unit of the GPCR-based chemical sensor.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

As used herein, “about,” “approximately,” and the like, when used in connection with a numerical variable, generally refers to the value of the variable and to all values of the variable that are within the experimental error (e.g., within the 95% confidence interval for the mean) or within .+−. 10% of the indicated value, whichever is greater.

As used herein, “control” is an alternative subject or sample used in an experiment for comparison purposes and included to minimize or distinguish the effect of variables other than an independent variable.

As used herein, “specifically binds” or “specific binding” refers to binding that occurs between such paired species such as enzyme/substrate, receptor/agonist or antagonist, antibody/antigen, lectin/carbohydrate, oligo DNA primers/DNA, enzyme or protein/DNA, and/or RNA molecule to other nucleic acid (DNA or RNA) or amino acid, which may be mediated by covalent or non-covalent interactions or a combination of covalent and non-covalent interactions. When the interaction of the two species produces a non-covalently bound complex, the binding that occurs is typically electrostatic, hydrogen-bonding, or the result of lipophilic interactions. Accordingly, “specific binding” occurs between a paired species where there is interaction between the two which produces a bound complex having the characteristics of an antibody/antigen, enzyme/substrate, DNA/DNA, DNA/RNA, DNA/protein, RNA/protein, RNA/amino acid, receptor/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

As used herein, “overexpressed” or “overexpression” refers to an increased expression level of an RNA or protein product encoded by a gene as compared to the level of expression of the RNA or protein product in a normal or control cell.

As used herein, “underexpressed” or “underexpression” refers to decreased expression level of an RNA or protein product encoded by a gene as compared to the level of expression of the RNA or protein product in a normal or control cell.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, gene deletion refers to a mutation introduced into the genome of an organism that completely or partially removes a physical portion of the nucleotide sequence for the gene to disrupt the production of a gene product generated from that gene or otherwise disrupts and/or ablates the production of the product of that gene. Deletions can be said to result in gene knockout or knockdown. Deletions can be homozygous (both or all copies deleted), heterozygous (only one or less than all copies deleted), or hemizygous.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid sequence” and “oligonucleotide” also encompasses a nucleic acid and polynucleotide as defined above.

As used herein, “DNA molecule” includes nucleic acids/polynucleotides that are made of DNA.

As used herein, “wild-type” is the typical form of an organism, variety, strain, gene, protein, or characteristic as it occurs in nature, as distinguished from mutant forms that may result from selective breeding or transformation with a transgene.

As used herein, “identity,” is a relationship between two or more polypeptide or polynucleotide sequences, as determined by comparing the sequences. In the art, “identity” also refers to the degree of sequence relatedness between polypeptide as determined by the match between strings of such sequences. “Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48:1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch (J. Mol. Biol., 1970, 48:443-453) algorithm (e.g., NBLAST, and XBLAST). The default parameters are used to determine the identity for the polypeptides or polynucleotides of the present disclosure.

As used herein, “heterologous” refers to compounds, molecules, nucleotide sequences (including genes), and polypeptide sequences (including peptides and proteins) that are different in both activity (function) and sequence or chemical structure. As used herein, “heterologous” can also refer to a gene or gene product that is from a different organism. for example, a human GPCR can be said to be heterologous when expressed in yeast.

As used herein, “homologue” refers to a polypeptide sequence that shares a threshold level of similarity and/or identity as determined by alignment of matching amino acids. Two or more polypeptides determined to be homologues are said to be homologues. Homology is a qualitative term that describes the relationship between polypeptide sequences that is based upon the quantitative similarity.

As used herein, “paralog” refers to a homologue produced via gene duplication of a gene. In other words, paralogs are homologues that result from divergent evolution from a common ancestral gene.

As used herein, “orthologues” refers to homologues produced by speciation followed by divergence of sequence but not activity in separate species. When speciation follows duplication and one homologue sorts with one species and the other copy sorts with the other species, subsequent divergence of the duplicated sequence is associated with one or the other species. Such species specific homologues are referred to herein as orthologues.

As used herein, “xenologs” are homologues resulting from horizontal gene transfer.

As used herein, “similarity” is a quantitative term that defines the degree of sequence match between two compared polypeptide sequences.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “culturing” refers to maintaining cells under conditions in which they can proliferate and avoid senescence as a group of cells. “Culturing” can also include conditions in which the cells also or alternatively differentiate.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof.

As used herein, “gene” refers to a hereditary unit corresponding to a sequence of DNA that occupies a specific location on a chromosome and that contains the genetic instruction for a characteristic(s) or trait(s) in an organism.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “plasmid” as used herein refers to a non-chromosomal double-stranded DNA sequence including an intact “replicon” such that the plasmid is replicated in a host cell.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “operatively linked” indicates that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and transcription control elements (e.g. promoters, enhancers, and termination elements), and/or selectable markers in an expression vector.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome.

As used herein, “transformation” or “transformed” refers to the introduction of a nucleic acid (e.g., DNA or RNA) into cells in such a way as to allow expression of the coding portions of the introduced nucleic acid.

As used herein, “stable expression,” “stable incorporation,” “stable transfection” and the like refer to the integration of an exogenous gene into the genome of a host cell, which can allow for long term expression of the exogenous gene.

As used herein, “transient expression,” “transient transfection,” and the like refer to the introduction of an exogenous gene into a host cell that does not result in stable incorporation of the gene into the host cell.

As used herein “chemical” refers to any molecule, compound, particle, or other substance that can be a substrate for a G-protein coupled receptor. As such, “chemical” can refer to nucleic acids, proteins, organic compounds, inorganic compounds, etc.

As used herein “biologically coupled” refers to the association of or interaction between two or more physically distinct molecules, groups of molecules compounds, organisms, or particles where the association is directly or indirectly mediated between the two or more physically distinct molecules, groups of molecules compounds, organisms or particles via a biologic molecule or compound. This can include direct binding between two biologic molecules and signal transduction pathways.

As used herein, “biological communication” refers to the communication between two or more molecules, compounds, or objects that is mediated by a biologic molecule or biologic interaction.

As used herein, “biologic molecule,” “biomolecule,” and the like refer to any molecule that is present in a living organism and includes without limitation, macromolecules (e.g. proteins, polysaccharides, lipids, and nucleic acids) as well as small molecules (e.g. metabolites and other products produced by a living organism).

As used herein, a “biologic interaction” refers to the interaction between two biomolecules.

As used herein, “regulation” refers to the control of gene or protein expression or function.

As used herein, “signaling molecule” refers to a molecule, such as a biomolecule, capable of producing a measurable signal when expressed. The signal can be qualitative or quantitative. The signal can be measured by any suitable techniques, which will be instantly appreciated by those of skill in the art.

As used herein, “promoter” refers to the DNA sequence(s) that control or otherwise modify transcription of a gene and can include binding sites for transcription factors, RNA polymerases, and other biomolecules and substances (e.g. inorganic compounds) that can influence transcription of a gene by interaction with the promoter. Typically these sequences are located at the 5′ end of the sense strand of the gene, but can be located anywhere in the genome.

As used herein, “native” refers to the endogenous version of a molecule or compound relative to the host cell or population being described.

As used herein, “non-naturally occurring” refers to a non-native version of a molecule or compound or non-native expression or presence of a molecule or compound within a host cell or other composition. This can include where a native molecule or compound is influenced to be expressed or present at a different location within a host, at a non-native period of time within a host, or is otherwise in an altered environment, even when considered within the host. Non-limiting examples include where a protein that is expressed only in the nucleus of a cell is expressed in the cytoplasm of the cell or when a protein that is only normally expressed during the embryonic stage of development is expressed during the adult stage.

As used herein, “encode” refers to the biologic phenomena of transcribing DNA into an RNA that, in some cases, can be translated into a protein product. As such, when a protein is said herein to be encoded by a particular nucleotide sequence, it is to be understood that this refers to this biologic relationship between DNA and protein. It is well established that RNA can be translated into protein based on the triplet code where 3 nucleotides represent an amino acid. This term also includes the idea that DNA can be transcribed into RNA molecules with biologic functions, such as ribozymes and interfering RNA species. As such, when a RNA molecule is said to be encoded by a particular nucleotide sequence it is to be understood that this is referring to the transcriptional relationship between the DNA and RNA species in question. As such “encoding nucleotide” refers to herein as the nucleotide which can give rise through transcription, and in the case of proteins, translation a functional RNA or protein.

As used herein, “fast maturing” refers to a signal molecule (e.g. a fluorescent protein) that can be measured, preferably within the linear range of the signal molecule, within about 0 to about 4 hours of initial contact of the signal molecule, a sensor system containing the signal molecule (such as those described herein), or sensor organism containing the signal molecule (such as those described herein), with a sample, substrate of the signaling molecule, aforementioned system or organism.

### GPCR-Based Chemical Biosensors

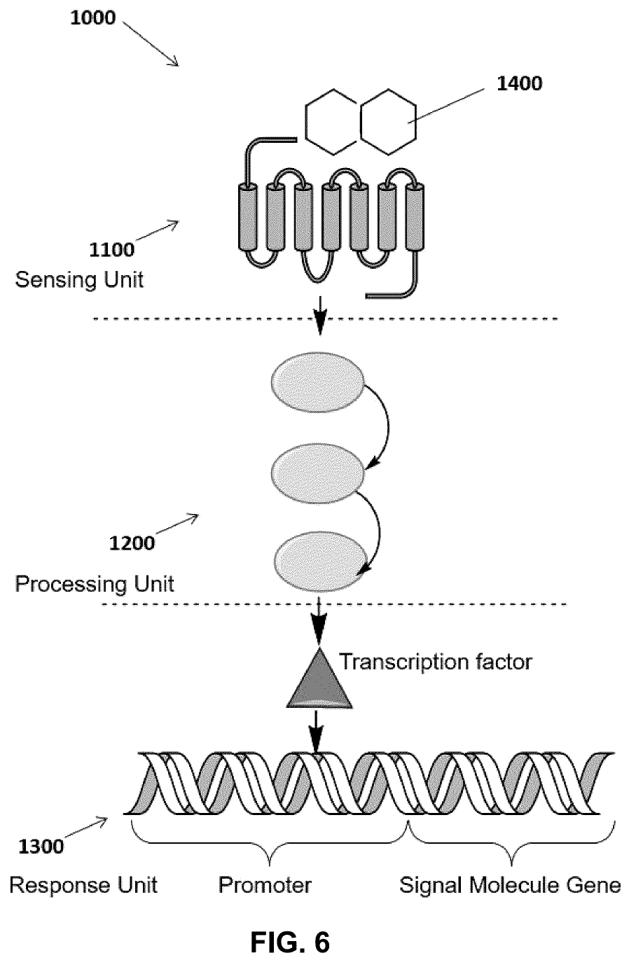

Described herein are GPCR-based chemical biosensors that can contain physically distinct components that are operatively coupled to each other and can detect a chemical and generate a signal indicating the presence or absence of a chemical. With the general description in mind, attention is direct to FIG. 6 which shows the features of a GPCR-based chemical sensor. The GPCR-based chemical sensors 1000 described herein can contain a sensing unit 1100, a processing unit 1200, and a response unit 1300, where the sensing unit 1100 can be biologically coupled to and/or in biologic communication with the processing unit 1200 and the processing unit can be biologically coupled to and/or in biologic communication with the response unit 1300.

The sensing unit 1100 can contain a GPCR that can bind to or otherwise interact with a chemical 1400. The sensing unit 1100 can biologically interact with processing unit 1200, which in turn can biologically interact with the response unit 1300. The biologic interaction between the different units of the GPCR-based chemical biosensors 1000 can be direct (i.e. no intermediate molecules, processes, and/or pathways involved in the biological interaction between one or more components of the interacting units) or indirect (i.e. involve one or more intermediate molecules, processes, and/or pathways in the biological interaction between one or more components of the interacting units, where the additional molecules, process and/or pathways are not part of the sensing unit, processing unit, or response unit).

In operation, a chemical 1400 can bind, unbind, or otherwise interact with the GPCR of the sensing unit 1100. Upon chemical interaction with the GPCR of the sensing unit 1100 the GPCR can biologically interact with the processing unit 1200. In some embodiments, chemical binding (or other interaction) with the GPCR of the sensing unit 1100 can stimulate, either directly or indirectly, a signal transduction pathway that is part of the processing unit 1200. The signal transduction pathway of the processing unit 1200 can then biologically interact with the response unit 1300, which can then generate or extinguish a signal. In some embodiments, the biological interaction between the processing unit 1200 and the response unit 1300 can be direct or indirect regulation of a signaling molecule gene promoter. In this way the processing unit 1200 can transmit a biological signal indicating the interaction of a chemical with the sensing unit 1100 to the response unit 1300, which can signal the presence (or absence) of a chemical 1400.

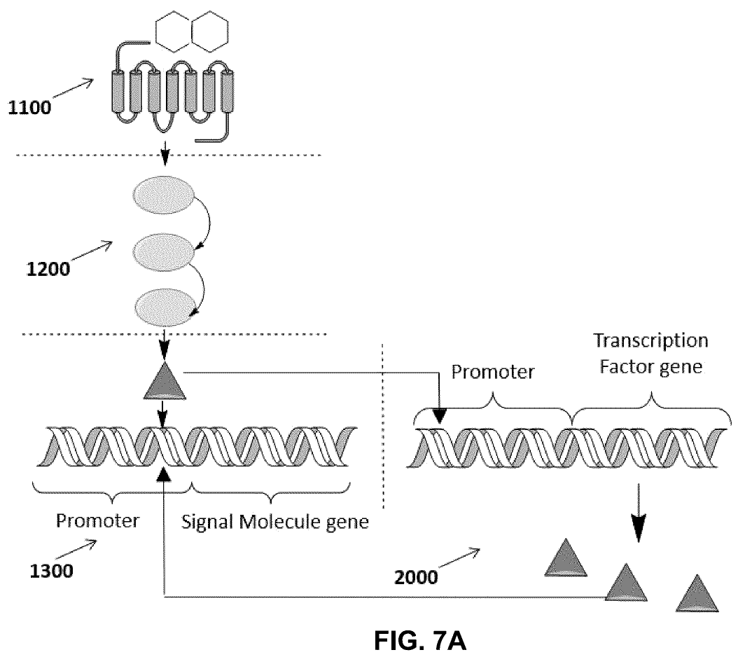

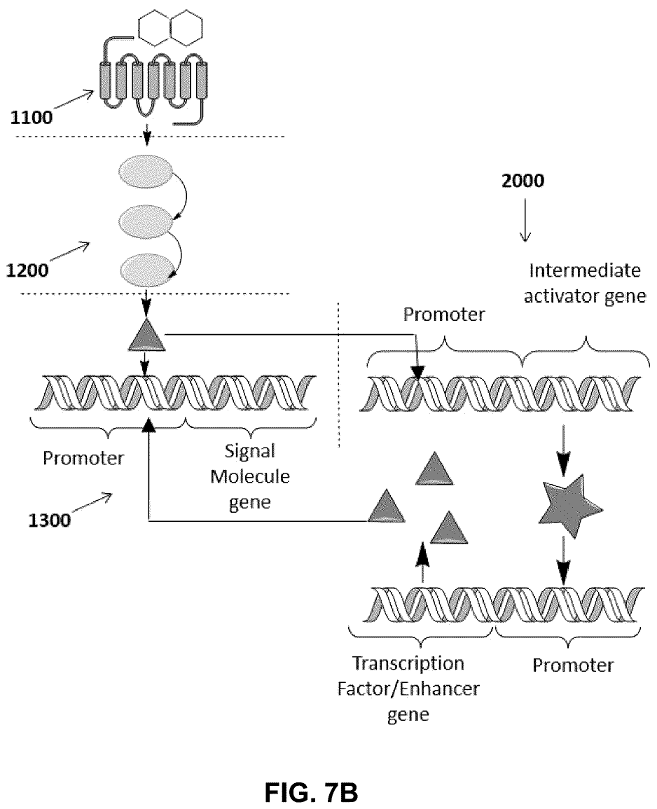

As shown in FIGS. 7A and 7B, the GPCR-based chemical biosensor 1000 can optionally contain an amplification unit 2000. The amplification unit 2000 can be configured to directly (FIG. 7A) or indirectly (FIG. 7B) amplify the signal generated by the response unit 1300. Generally, the amplification unit 2000 can act as a feed forward loop that stimulates increased signal production from the response unit 1300 when the response unit is biologically acted upon, either directly or indirectly, by the processing unit. 1200. In short, the amplification unit can autoamplify the signal from the response unit 1300 in response to a chemical 1400 binding, unbinding, or otherwise interacting with the GPCR of the sensing unit 1100. The mechanism by which amplification can occur is described in greater detail elsewhere herein.

The physically distinct components can be expressed within a whole cell, such as a yeast cell. In other embodiments, the physically distinct components can be expressed in a synthetic in vitro system. The physically distinct components can be considered modular components where each one can be independently manipulated and changed without alteration of the other components. This modular configuration can allow for efficient and rapid tuning and customization of system based on the desired sensing and signaling capabilities of the GPCR-based chemical biosensor. The individual modular components are discussed in further detail below.

### Sensing Unit

As shown in FIG. 6, the sensing unit 1100 can contain a GPCR with a mutated or truncated C-terminus. The GPCR can be a native GPCR. In some embodiments, the GPCR can be a non-naturally occurring GPCR, which includes but is not limited to, recombinant and other engineered GPCRs. In some embodiments, the GPCR can be a heterologous GPCR, a homologous GPCR, an orthologous GPCR, or a paralogous GPCR. The GPCR can be a GPCR in the family of Rhodopsin-like GPCRs, Secretin receptor GPCRs, metabotropic glutamate/pheromone GPCRs, fungal mating pheromone GPCRs, Cyclic AMP GPRCs, or Frizzled, Smoothened GPCRs. In some embodiments, the GPCR is codon optimized for the organism in which the GPCR-based biosensor is to be expressed in. In some embodiments, the GPCR can be GPCR40 (also referred to herein as GPR40). In other embodiments, the GPCR can be OR1G1 or any other olfactory receptor GPCR. In other embodiments, the GPCR can be M3 muscarinic receptor, D2S Dopamine receptor, Beta2 Adrenergic receptor, Beta Alanine receptor, Nicotinamide receptor, OR56, Geosmin GPCR, melatonin receptor (mel1a), or AT1R. In further embodiments, the GPCR can be STE2 GPCR or STE3 GPCR.

The sensing unit 1100 and/or the GPCR of the sensing unit 1100 can be configured to biologically interact with the processing unit 1200. The GPCR can be configured to interact with one or more signal transduction pathways within the host cell (i.e. the cell in which the GPCR is expressed in). GPCRs contain three subunits (typically denoted Ga, GB/GA) that interact with each other either by the subunits associating with one another upon binding/unbinding a substrate (e.g. chemical) or one or more subunits disassociating from the other subunit(s) upon binding/unbinding a substrate. The disassociation or association of one or more subunits of the GPCR can stimulate or inactivate a downstream signal transduction pathway present in a host cell or in vitro environment. In some embodiments, the signal transduction pathway can be part of the processing unit of the GPCR-based chemical biosensor. In other embodiments, this signal transduction pathway is an intermediate between the sensing unit 1100 and the processing unit 1200. It will be appreciated by those of ordinary skill in the art that the signal transduction pathway will vary based on the GPCR employed in the sensing unit 1100 and the host cell. The signal transduction pathway can be a MAPK pathway, adenylyl cyclase pathway, phospholipase C pathway, arachidonic acid pathway, cyclic AMP (CAMP) pathway, RhoGEF signaling pathways, ion channels (e.g. G-protein-regulated rectifying K+ channels, P/Q- and N-type voltage gated channels, and posphoinositide-3-kinase pathways. In some embodiments, the GPCR can directly signal the processing unit through signaling β-arrestin, G protein-coupled receptor kinases, and tyrosine kinases (e.g. proto-oncogene tyrosine-protein kinase Src).

The GPRC of the sensing unit 1100 can be configured to bind any desired chemical. The GPCR can naturally bind a chemical of interest or can be modified to have improved or otherwise altered binding characteristics (e.g. bind a substrate that would not naturally bind to the GPCR). In some embodiments, the GPCR can bind a medium chain (i.e. a C8-C14) fatty acid. In some embodiments, the chemical can be C10 fatty acid.

### Processing Unit

The GPCR-based chemical biosensor 1000 described herein can contain a processing unit 1200. The processing unit 1200 can include one or more endogenous, synthetic, or otherwise modified signal transduction pathway. In synthetic or otherwise modified signal transduction pathways at least one molecule involved in the signal transduction pathway can be recombinant, or otherwise non-natural. The signal transduction pathway can be a MAPK pathway, adenylyl cyclase pathway, phospholipase C mediated pathway (e.g. inositol 1,4,5-triposphate (IP3)/Diacyl glycerol (DAG) pathway), arachidonic acid pathway, cyclic AMP (cAMP) pathway, RhoGEF signaling pathways, ion channels (e.g. G-protein-regulated rectifying K+ channels, P/Q- and N-type voltage gated channels, posphoinositide-3-kinase pathways, β-arrestin, G protein-coupled receptor kinases, histidine-specific protein kinase mediated pathways, tyrosine kinase mediate pathways, AKT pathways FAK mediated pathways, GSK3β pathways. In some embodiments, the processing unit 1200 can contain molecules within the mating pathway of yeast. In other words, the GPCR of the sensing unit 1100 can be configured to stimulate molecules in the mating pathway of yeast (e.g. Ste4, GPA1, Ste20, Ste5, Ste11, Ste7, and/or Fus3).

The signal transduction pathway of the processing unit 1200 can regulate one or more transcription factors. Regulation of transcription factors can include, but is not limited to, activation or suppression of transcription factors. One of ordinary skill in the art will appreciate the myriad of ways activation or suppression of a transcription factor(s) can occur and all are within the spirit and scope of this description. The transcription factor can be native to the host cell. In other embodiments, the transcription factor is a synthetic transcription factor that is not native to the host cell or the signaling pathway employed by the processing unit 1200. In some embodiments, the transcription factor is Ste12. In other embodiments, the transcription factor is a synthetic transcription factor including, but not limited to, STF1 (a transcription factor composed of the STE12 phosphorylation domain and the Gal4 activation and DNA binding domains (Pi, H. W., Chien, C. T., and Fields, S. (1997). Transcriptional activation upon pheromone stimulation can be mediated by a small domain of Saccharomyces cerevisiae Ste12p, Mol Cell Biol 17, 6410-6418.)), STF2 (a transcription factor composed of the STE12 phosphorylation domain, the synthetic B42 activation domain and the bacterial LexA DNA binding domain (Golemis, E. A., and Brent, R. (1992) Fused Protein Domains Inhibit DNA-Binding by Lexa, Mol Cell Biol 12, 3006-3014 and Peralta-Yahya, P., Carter, B. T., Lin, H. N., Tao, H. Y., and Comish, V. W. (2008) High-Throughput Selection for Cellulase Catalysts Using Chemical Complementation, J Am Chem Soc 130, 17446-17452)), STF3 (a transcription factor composed of the CRE protein activation and phosphorylation domain with the Gal4 DNA binding domain), STF4 (a transcription factor composed of the CRE protein activation and phosphorylation domain and the LexA DNA binding domain). The synthetic transcription factor can be configured to interact with an endogenous or a synthetic promoter.

### Response Unit

The GPCR-based chemical biosensor 1000 can contain a response unit 1300. The response unit can contain a signal molecule promoter operatively coupled to a signal molecule gene, where the signal molecule gene encodes or otherwise (e.g. by activating other pathways in the cell that results in the production of a gene product, such as a protein that can be measured) generates a signal molecule. The promoter can be configured to stimulate or extinguish transcription of the signal molecule gene (and subsequent production of the signal molecule) upon binding or unbinding of a transcription factor (such as one stimulated by the processing unit 1200). In this way, as signal (either appearance or disappearance of the signal molecule) can be generated by the GPCR-based chemical biosensor 1000 in response to binding, unbinding, or other interaction of a chemical 1400 with the GPCR of the sensing unit 1100.

The transcription factor can be native to the host cell or synthetic. In other embodiments, the transcription factor is a synthetic transcription factor that is not native to the host cell or the signaling pathway employed by the processing unit 1200. In some embodiments, the transcription factor is Ste12. In other embodiments, the transcription factor is a synthetic transcription factor including, but not limited to, STF1 (a transcription factor composed of the STE12 phosphorylation domain and the Gal4 activation and DNA binding domains (Pi, H. W., Chien, C. T., and Fields, S. (1997). Transcriptional activation upon pheromone stimulation can be mediated by a small domain of Saccharomyces cerevisiae Ste12p, Mol Cell Biol 17, 6410-6418.)), STF2 (a transcription factor composed of the STE12 phosphorylation domain, the synthetic B42 activation domain and the bacterial LexA DNA binding domain (Golemis, E. A., and Brent, R. (1992) Fused Protein Domains Inhibit DNA-Binding by Lexa, Mol Cell Biol 12, 3006-3014 and Peralta-Yahya, P., Carter, B. T., Lin, H. N., Tao, H. Y., and Comish, V. W. (2008) High-Throughput Selection for Cellulase Catalysts Using Chemical Complementation, J Am Chem Soc 130, 17446-17452)), STF3 (a transcription factor composed of the CRE protein activation and phosphorylation domain with the Gal4 DNA binding domain), STF4 (a transcription factor composed of the CRE protein activation and phosphorylation domain and the LexA DNA binding domain). The transcription factor can be directly stimulated by the processing unit or can be the product of another signal transduction pathway stimulated by the processing unit (via a transcription factor or other mode of pathway stimulation).

In some embodiments the transcription factor can have a sequence about 90% to 100% identical to SEQ ID NOS: 27-29.

STF1 sequence
(SEQ ID NO: 27)
ATGAAGCTACTGTCTTCTATCGAACAAGCATGCGATATTTGCCGA
CTTAAAAAGCTCAAGTGCTCCAAAGAAAAACCGAAGTGCGCCAAG
TGTCTGAAGAACAACTGGGAGTGTCGCTACTCTCCCAAAACCAAA
AGGTCTCCGCTGACTAGGGCACATCTGACAGAAGTGGAATCAAGG
CTAGAAAGACTGGAACAGCTATTTCTACTGATTTTTCCTCGCGAA
GACCTTGACATGATTTTGAAAATGGATTCTTTACAGGATATAAAA
GCATTGTTAACAGGATTATTTGTACAAGATAATGTGAATAAAGAT
GCCGTCACAGATAGATTGGCTTCAGTGGAGACTGATATGCCTCTA
ACATTGAGACAGCATAGAATAAGTGCGACATCATCATCGGAAGAG
AGTAGTAACAAAGGTCAAAGACAGTTGACTGTATCTAGACCATCT
AGTACAACAAAATCAGATAATTCGCCTCCAAAATTAGAAAGCGAG
AATTTTAAGGATAATGAGTTGGTAACAGTAACTAATCAGCCGCTT
TTAGGCGTTGGCCTCATGGATGACGATGCGCCAGAATCCCCCTCT
CAAATTAATGATTTTATTCCTCAGAAATTGATTATAGAACCCAAT
ACTCTCGAATTGAATGGTCTCACAGAAGAAACGCCTCATGACTTA
CCCAAGAATACCGCTAAGGGCAGAGACGAAGAAGATTTTCCTCTC
GACTATTTTCCTGTATCTGTTGAATACCCTACGGAGGAAAATGCG
TTTGATCCGTTCCCTCCACAGGCTTTTACGCCAGCTGCCCCTTCC
ATGCCTATTTCCTATGATAACGTGAATGAAAGGGATTCTATGCCC
GTTAATTCTCTTCTTAATAGATACCCCTATCAGTTATCAGTGGCA
CCCACTTTCCCAGTGCCACCATCATCATCGAGGCAACATTTTATG
TATCCTTACGACGTTCCAGATTATGCTATTGACTCTGCAGCTCAT
CATGATAACTCCACAATTCCGTTGGATTTTATGCCCAGGGATGCT
CTTCATGGATTTGATTGGTCTGAAGAGGATGACATGTCGGATGGC
TTGCCCTTCCTGAAAACGGACCCCAACAATAATGGGTTCTAA.
STF2 sequence
(SEQ ID NO: 28)
ATGGGTGCTCCACCTAAGAAGAAAAGAAAGGTTGCCAAAGCTTTG
ACTGCCAGACAACAAGAAGTCTTCGATTTGATTAGAGATCATATT
TCTCAAACTGGTATGCCACCAACTAGAGCTGAAATTGCTCAAAGA
TTGGGTTTCAGATCTCCAAACGCCGCTGAAGAACACTTGAAAGCT
TTGGCTAGAAAGGGTGTCATTGAAATTGTTTCTGGTGCTTCTAGA
GGTATTAGATTGTTGCAAGAAGAAGAAGAAGGTTTGCCATTGGTT
GGTAGAGTCGGTAGACCATCTTCTACTACTAAATCTGATAACTCT
CCACCAAAGTTGGAATCTGAAAACTTCAAAGATAACGAATTGGTT
ACTGTTACAAATCAACCATTGTTAGGTGTCGGTTTGATGGATGAC
GATGCTCCAGAATCTCCTTCTCAAATTAACGATTTCATTCCACAA
AAGTTGATTATTGAACCAAACACTTTGGAATTGAACGGTTTGACT
GAAGAAACTCCACACGATTTGCCAAAGAATACTGCCAAAGGTAGA
GATGAGGAAGACTTCCCATTGGATTACTTTCCAGTTTCTGTCGAA
TATCCAACTGAAGAAAACGCTTTCGATCCATTTCCACCACAAGCC
TTTACTCCAGCTGCACCATCTATGCCAATTTCTTACGATAACGTT
AATGAAAGAGATTCTATGCCAGTCAACTCATTGTTGAATAGATAC
CCATATCAATTGTCTGTTGCTCCAACTTTCCCAGTTCCTCCATCT
TCTTCAAGACAACACTTTATGGGTATTAACAAGGATATTGAGGAA
TGTAATGCCATCATTGAACAATTCATCGATTACTTGAGAACTGGT
CAAGAAATGCCAATGGAAATGGCCGATCAAGCCATTAACGTTGTC
CCAGGTATGACTCCAAAGACTATTTTGCACGCTGGTCCACCAATT
CAACCAGATTGGTTGAAATCTAACGGTTTCCACGAAATTGAAGCT
GATGTCAATGACACATCTTTGTTATTGTCTGGTGATGCCTCTTAA.
STF3 sequence
(SEQ ID NO: 29)
ATGACTATGGATTCTGGTGCTGATAATCAACAATCTTCTTGTAAA
GATTTGAAAAGATTGTTTTCTGGTACTCAAATTTCTACTATTGCT
GAATCTGAAGATTCTCAAGAATCTGTTGATTCTGTTACTGATTCT
CAAAAAAGAAGAGAAATTTTGTCTAGAAGACCATCTTATAGAAAA
ATTTTGAATGATTTGTCTTCTATTGAACAAGCTTGTGATATTTGT
AGATTGAAAAAATTGAAATGTTCTAAAGAAAAACCAAAATGTGCT
AAATGTTTGAAAAATAATTGGGAATGTAGATATTCTCCAAAAACT
AAAAGATCTCCATTGACTAGAGCTCATTTGACTGAAGTTGAATCT
AGATTGGAAAGATTGGAACAATTGTTTTTGTTGATTTTTCCAAGA
GAAGATTTGGATATGATTTTGAAAATGGATTCTTTGCAAGATATT
AAAGCTTTGTTGACTGGTTTGTTTGTTCAAGATAATGTTAATAAA
GATGCTGTTACTGATAGATTGGCTTCTGTTGAAACTGATATGCCA
TTGACTTTGAGACAACATAGAATTTCTGCTACTTCTTCTTCTGAA
GAATCTTCTAATAAAGGTCAAAGACAATTGACTGTTTCTATTGAT
TCTGCTGCTCATCATGATAATTCTACTATTCCATTGGATTTTATG
CCAAGAGATGCTTTGCATGGTTTTGATTGGTAA.

The signal molecule promoter can be a native promoter in the host cell. Suitable native promoters include without limitation those involved in the yeast mating pathway (e.g. P_{Fig1}, P_{Fus1}, P_{Fig3}, P_{Fig2}, P_{Fig4}). The signal molecule promoter can be a synthetic promoter. Suitable synthetic promoters include without limitation PGal4 (5×), which is described in greater detail elsewhere herein, and contains five Gal4 binding sites and PLexA (4×), which is described in greater detail elsewhere herein, and contains 4 LexA binding sites. In some embodiments, the synthetic promoter can have a nucleotide sequence about 90% to 100% identical to any one of SEQ ID Nos: 30-32.

PGal4(5x):
(The underlined ATG is the start codon)
(SEQ ID: NO: 30)
CCGAGCTCTTACGCGGGTCGAAGCGGAGTACTGTCCTCCGAGTGG
AGTACTGTCCTCCGAGCGGAGTACTGTCCTCCGAGTCGAGGGTCG
AAGCGGAGTACTGTCCTCCGAGTGGAGTACTGTCCTCCGAGCGGA
GTACTGTCCTCCGAGTCGACTCTAGAGGGTATATAATG.
PLexA(4x):
(The underlined ATG is the start codon)
(SEQ ID: NO: 31)
CCGAGCTCTTACGCGGGTCGAAGTGCTGTATATACTCACAGCAAG
TGGAGTACTGTCCTCCGAGAACTGTATATACACCCAGGGAGTCGA
GGGTCGAAGTACTGTATGAGCATACAGTAAGTGGAGTACTGTCCT
CCGAGAACTGTATATAAATACAGTTAGTCGACTCTAGAGGGTATA
TAATG.
PCre
(SEQ ID NO: 32)
TCCTGGAAGTCTCATGGAGATTATACTTTATGCACCAGACAGTGA
CGTCAGCTGCCAGATCCCATGGCCGTCATACTGTGACGTCTTTCA
GACACCCCATTGACGTCAATGGGAGAACTTTAGTATCCGTTTAGC
TAGTTAGTACCTTTGCACGGAAATGTATTAATTAGGAGTATATTG
AGAAATAGCCGCCGACAAAAAGGAAGTCTCATAAAAGTGTCTAAC
AGACAATTAGCGCAATAAGAAGAAAGAAAACGGATTGAAGTTGAG
TCGAGAATAATATGGCACCCAGAAAACGCTTTAGGCTACTCGAAT
TAGGGTCACCAATG.
In some embodiments, the promoter can be or
include a repressor element. In some embodiments
the repressor can consist of or include a sequence
about 90% to about 100% identical to SEQ ID NO: 33
and/or SEQ ID NO: 34.
pGal4(5x) repressor
(SEQ ID NO: 33)
TCGACTCTAGAGGGTATATACCGAGCTCTTACGCGGGTCGAAGCG
GAGTACTGTCCTCCGAGTGGAGTACTGTCCTCCGAGCGGAGTACT
GTCCTCCGAGTCGAGGGTCGAAGCGGAGTACTGTCCTCCGAGTGG
AGTACTGTCCTCCGAGCGGAGTACTGTCCTCCGAGGGATCCATG.
pLexA(4x) repressor
(SEQ ID NO: 34)
CCGAGCTCTTACGCGGGTCGAAGTGCTGTATATACTCACAGCAAG
TGGAGTACTGTCCTCCGAGAACTGTATATACACCCAGGGAGTCGA
GGGTCGAAGTACTGTATGAGCATACAGTAAGTGGAGTACTGTCCT
CCGAGAACTGTATATAAATACAGTTAGTCGACTCTAGAGGGTATA
TAATGATG.

The signal molecule promoter can be operatively coupled to a signal molecule gene, which can encode a suitable signal molecule. Suitable signal molecules include without limitation, a fluorescent protein, β-galactosidase protein, a luciferase protein, and chloramphenicol acetyltransferase, antibiotic resistance markers such as KanMX4, auxotrophic genes such as His3, Ura3, TRp1, Leu2 which enable selections and counter selections, a biosynthetic gene or pathway that results in the production of a colorimetric or fluorescent compound, such as lycopene, indigo or violacein, a synthetic RNA, a synthetic DNA or a ribozyme Suitable fluorescent proteins include without limitations, green fluorescent proteins and enhanced green fluorescent proteins, yellow fluorescent proteins and enhanced yellow fluorescent proteins, blue fluorescent proteins and enhanced blue fluorescent proteins, cyan fluorescent proteins and enhanced cyan fluorescent proteins, orange fluorescent proteins and enhanced orange fluorescent proteins, and red fluorescent proteins and enhanced red fluorescent proteins. Fluorescent proteins are generally known in the art and are commercially available. All of these are within the scope and spirit of the present disclosure. The signal molecule gene can be codon optimized for expression within the particular host cell. In some embodiments, the signal molecule is a fast maturing signal molecule.

### Amplification Unit

As shown in FIGS. 7A and 7B, the GPCR-based chemical biosensor 1000 can optionally contain an amplification unit 2000. The amplification unit 2000 can be configured to directly (FIG. 7A) or indirectly (FIG. 7B) amplify the signal generated by the response unit 1300. Generally, the amplification unit 2000 can act as a feed forward loop that stimulates increased signal production from the response unit 1300 when the response unit is biologically acted upon, either directly or indirectly, by the processing unit. 1200. In short, the amplification unit can autoamplify the signal from the response unit 1300 in response to a chemical 1400 binding, unbinding, or otherwise interacting with the GPCR of the sensing unit 1100.

As shown in FIG. 7A the amplification unit 2000 can be configured to directly amplify the signal generated by the response unit 1300. The amplification unit can contain an amplification unit promoter that can be operatively coupled to a transcription factor gene. The product(s) of the transcription factor gene can be a suitable transcription factor. In operation, the transcription factor stimulated by the processing unit 1200 can bind the signal molecule gene promoter of the sensing unit 1300 and the promoter of the amplification unit 2000. The promoter of the amplification unit 2000 can drive gene expression of a transcription factor that can bind also bind or otherwise interact with the signal molecule promoter of the response unit 1300 to drive gene expression of the signal molecule and generate additional signal molecules in a feed forward fashion. Insofar as additional signal molecules can be generated without additional input stimulation from the sensing unit 1100, the signal from the GPCR-based chemical biosensor can be amplified.

Suitable transcription factors produced by the amplification unit 2000 can be any transcription factor configured to bind or otherwise active the signal molecule gene promoter of the response unit 1300 and generate an upregulation in gene expression of the signal molecule gene. In some embodiments, the transcription factor produced by the amplification unit 2000 can be the same transcription factor produced or stimulated by the processing unit 1200. In some embodiments, the transcription factor produced by the amplification unit 2000 can be Ste12, STF1, or STF2.

Suitable promoters for the amplification unit 2000 can include native and synthetic promoters. The amplification unit promoter can be a native promoter in the host cell. Suitable native promoters include without limitation those involved in the yeast mating pathway. The amplification unit promoter can be a synthetic promoter. Suitable synthetic promoters include without limitation PGal4 (5×), which is described in greater detail elsewhere herein, and contains five Gal4 binding sites and PLexA (4×), which is described in greater detail elsewhere herein, and contains 4 LexA binding sites. In some embodiments, the synthetic promoter can have a nucleotide sequence identical to any one of SEQ ID Nos: 30-32.

As shown in FIG. 7B, the optional amplification unit 2000 can indirectly amplify the signal generated by the GPCR-based chemical biosensor. In these embodiments, the amplification unit 2000 can contain a first amplification unit promoter operatively coupled to an intermediate activator gene. The intermediate activator gene can encode for a suitable intermediate molecule that is capable of binding a second amplification unit promoter that is operatively coupled to a transcription factor gene. Any promoter described herein or any other native promoter can be used as a promoter in the amplification unit. The promoter can be operatively coupled to the transcription factor gene. The second amplification unit promoter and the transcription factor gene can be as described with respect to the amplification unit promoter and transcription factor gene and gene product(s). Any promoter described herein or any other native promoter can be used as a promoter in the amplification unit. The promoter can be operatively coupled to the transcription factor gene or intermediate activator gene.

In operation, the transcription factor produced by the processing unit 1200 can bind or otherwise activate both the signal molecule promoter of the response unit 1300 and the first amplification unit promoter. When the first amplification unit promoter is activated it can drive expression of the intermediate activator gene and thus production of a suitable intermediate activator molecule (e.g. another transcription factor or other protein involved in up-regulation of genes, particularly those that are part of the amplification unit). The intermediate activator molecule can then bind or otherwise activate the second amplification unit promoter and thus stimulate production of a transcription factor that can bind or otherwise interact with the signal molecule promoter of the response unit 1300 to drive gene expression of the signal molecule and generate additional signal molecules in a feed forward fashion. Insofar as additional signal molecules can be generated without additional input stimulation from the sensing unit 1100, the signal from the GPCR-based chemical biosensor can be amplified.

### Host Cells

The sensing unit, processing unit, and/or the response unit can be expressed or otherwise contained within a single host cell. The host cell can be eukaryotic or prokaryotic. In some embodiments, the host cell can be a mammalian cell, a fungal cell, or a bacterial cell. In some embodiments the host cell is a yeast cell. Suitable yeast species for the host cell include but are not limited to S. cerevisiae, Pichia Pastoris, Saccharomyces Pombe. Suitable strains of S. cerevisiae include, but are not limited to the W303 strain (ATCC), PPY62, PPY58, PPY140, and PPY161. The GPCR-based chemical biosensors can be introduced into the host cell via a single or multiple plasmid system or integrated into the genome. The GPCR-based chemical biosensor or can be stably or transiently expressed within the host cell. In some embodiments, the host cell is different from a producer cell (i.e., a cell that produces a chemical to be detected by the GPCR-based chemical biosensor). The GPCR-based chemical sensors can be used to evolutionary engineer or high-throughput engineering chemical-producing microbes using medium-throughput methods (e.g. 96-well plate), or high-throughput methods (e.g. microfluidic chip).

### Systems and Methods of Using the GPCR-Based Chemical Biosensors

Also described herein are systems and methods of using the GPCR-based chemical biosensors. As described above the modular components of the GPCR-based chemical biosensors can be expressed within a host cell (also referred to herein as a sensor cell or sensor strain). The host cell can then be used in a method to sense a chemical (which includes proteins) of interest. The method can include incubating a host cell containing a GPCR-based chemical biosensor as described herein in a solution or environment containing a sample, a cell, or other composition to be analyzed for a period of time. After the period of time, a suitable assay or other suitable measurement technique can be performed to measure the amount of signal molecule produced by the GPCR-based chemical biosensor. One of skill in the art will appreciate that the particular assays or measurement technique used will depend on the type of signaling molecule produced. Suitable assays and measurement techniques include, but are not limited to, flow cytometry, FACS, luciferase assays (single and dual), β-galactosidase assays, microtiter plate reader, and CAT assays, antibiotic selection, auxotrophic forward and counter selection. Other assays and techniques will be readily appreciated by those of ordinary skill in the art.

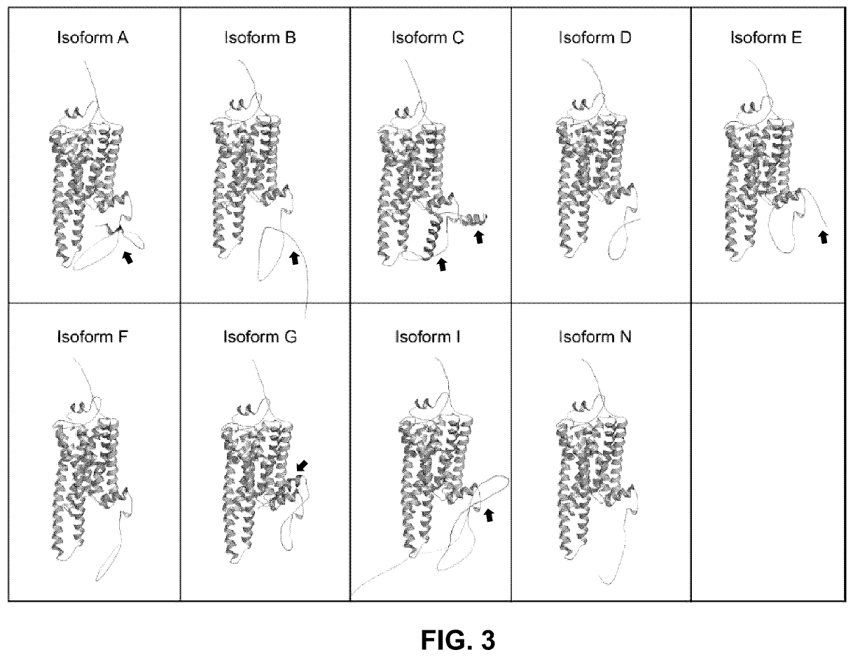

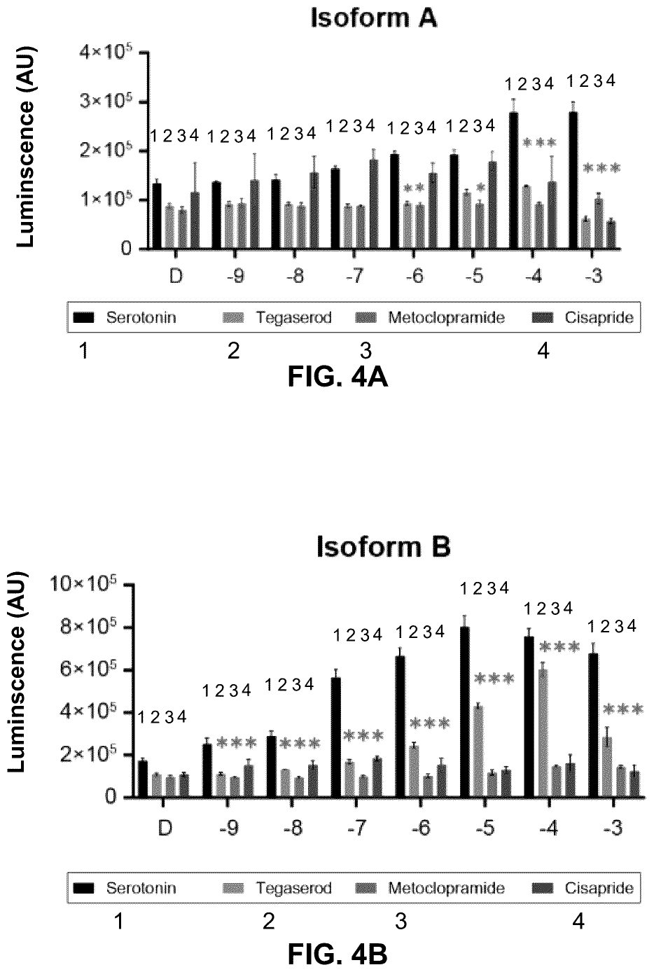

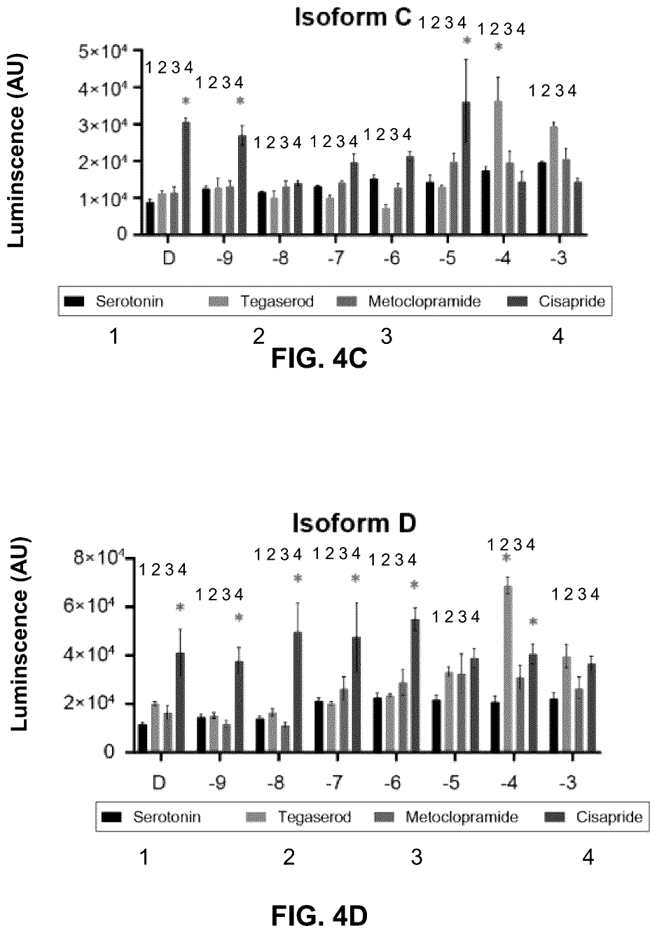

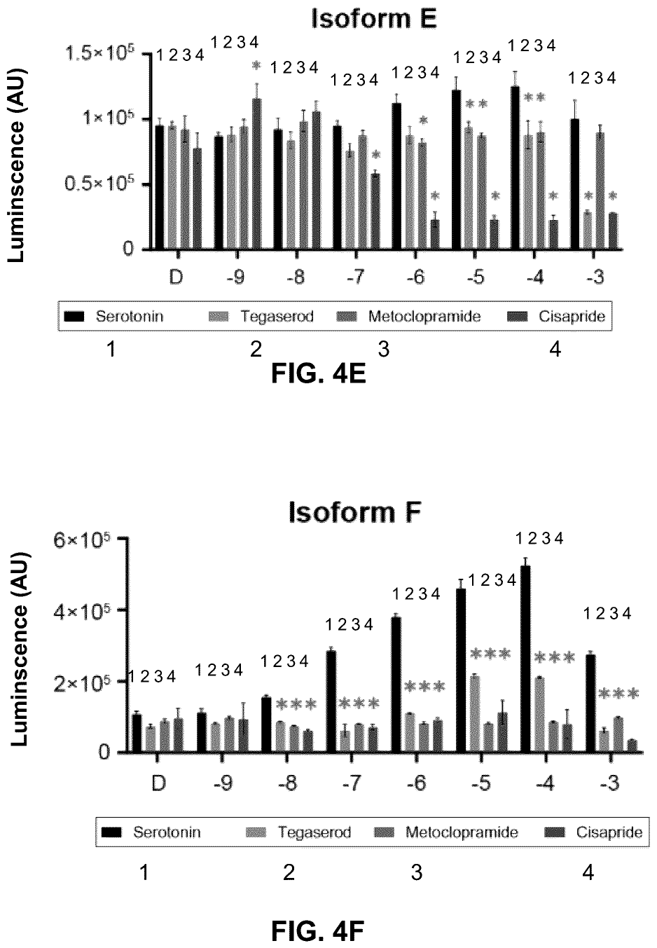

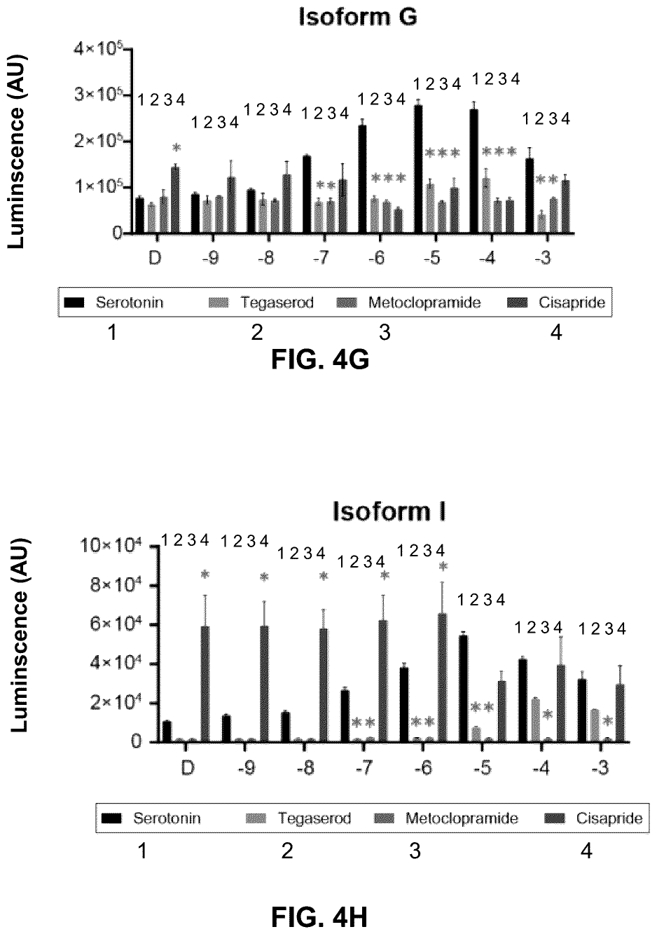

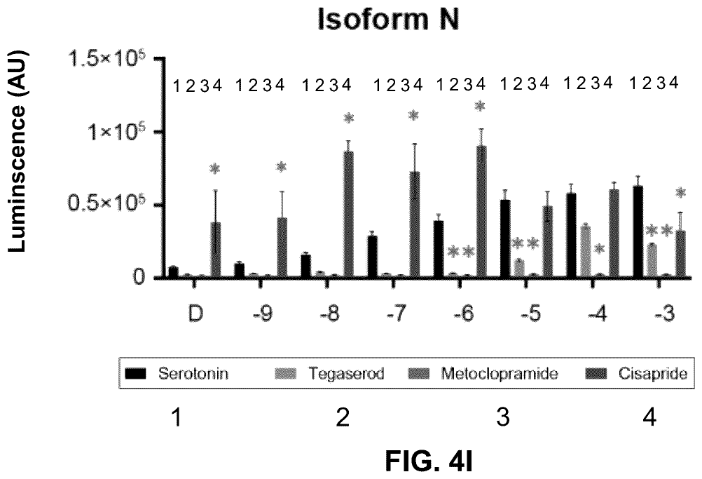

In some embodiments, the sensor cell or strain can be used to detect a medium chain fatty acid in a sample. In other embodiments the sensor strain can be used to detect production of a desired chemical (which includes proteins) such as a medium chain fatty acid, from a producer cell. These can be accomplished in a low-throughput or medium through-put fashion. FIG. 3 of U.S. Provisional Patent Application Ser. No. 63/749,312 (not shown; see also S1 of Appendix A of U.S. Provisional Patent Application Ser. No. 63/749,312; see also FIG. S1 of Marquez-Gomez P L, Damiano S R, Torp L R, Peralta-Yahya P. Modulating the Properties of GPCR-Based Sensors Via C-Terminus Isoforms. ACS Synth Biol. 2025 May 16; 14 (5): 1853-1860. doi: 10.1021/acssynbio.4c00847. Epub 2025 Apr. 25. PMID: 40279474; PMCID: PMC12090342., which is incorporated by reference as if fully set forth herein) contains sequence alignment of 5-HT1 isoforms studied to date. N-term: N-terminus. TM: Transmembrane domain. ICL: Intracellular Loop. ECL: Extracellular Loop. C-term; C-terminus. Sequence alignment done using Clustal Omega. FIG. 4 U.S. of Provisional Patent Application Ser. No. 63/749,312 (not shown; see also FIG. S2 of Appendix A of U.S. Provisional Patent Application Ser. No. 63/749,312; see also FIG. S2 of Marquez-Gomez P L, Damiano S R, Torp L R, Peralta-Yahya P. Modulating the Properties of GPCR-Based Sensors Via C-Terminus Isoforms. ACS Synth Biol. 2025 May 16; 14 (5): 1853-1860. doi: 10.1021/acssynbio.4c00847. Epub 2025 Apr. 25. PMID: 40279474; PMCID: PMC12090342, which is incorporated by reference as if fully set forth herein) contains sequence alignment of 5-HTR₄ C-terminus isoforms studied in this work. FIG. 3 shows alphaFold structural predictions for 5-HTR4 c-terminus isoforms A, B, C, D, E, F, G, I and N. SequencesN-term: N-terminus. TM: Transmembrane domain. ICL: Intracellular Loop. ECL: Extracellular Loop. C-term; C-terminus. Sequence alignment done using Clustal Omega.

After the first period of time, a sensor cell(s) can be added to the wells as desired and incubated for a second period of time to allow for interaction, such as binding, between the chemical produced by the producer cell in each well and the GPRC of sensor cell present in the same well. The second period of time can be an amount of time sufficient for biosensor production. The second period of time can range from about 0 to about 96 hours, about 96 to about 72 hours, about 72 to about 60 hours, about 60 hours to about 48 hours, about 48 hours to about 36 hours, about 36 hours to about 24 hours, about 24 hours to about 12 hours, about 12 hours to about 6 hours, about 4 hours to about 6 hours, about 2 hours to 4 hours, and about 0 to about 2 hours. In some embodiments, particularly those when a fast maturing signaling molecule is used, the second period of time can range from about 0 hours to about 3 hours. In other embodiments, the second period of time can be about 4 hours. In further embodiments, the second period of time can be about 1 hour.

After the second period of time, a suitable assay or measurement technique can be performed to measure the amount of signal molecule produced from each well. This can allow for determining which producing cells produced the chemical of interest. In embodiments, where the signal measurement assay/technique can allow for quantification of the amount of signal produced, it can be determined which producing cells produced the most chemical. Such techniques that can allow for quantification include flow cytometry, FACS, luciferase assays, β-galactosidase assays, microtiter plate reader, antibiotic selection, auxotrophic forward and counter selection and CAT assays. Others will be appreciated by those of skill in the art. In this way, one can select which producing strain is desired based on the determination of their ability to produce (or not produce) a particular chemical.

In some embodiments the sensor cells as described herein can be used in any of the methods previously described to detect a fatty acid. In some embodiments, the fatty acid is a medium chain fatty acid. In some embodiments, the medium chain fatty acid is a C10 fatty acid.

In some embodiments, the GPCR-based chemical biosensor or assay using the GPCR-based chemical biosensor can have a linear range of detection of up to about 250 μM. The GPCR-based chemical biosensor can have a linear detection range of about 500 μM or greater. In some embodiments the linear detection range can be from about 0 to 1 μM or any range within that. In some embodiments, the GPCR-based chemical biosensor or assay using the GPCR-based chemical biosensor can have a linear range of detection of up to about 500 μM. In some embodiments, the GPCR-based chemical biosensor or assay using the GPCR-based chemical biosensor can have a linear detection range of about 34 μM to about 250 μM. The GPCR-based chemical biosensor or assay using the GPCR-based chemical biosensor can have a linear detection range of about 110 μM to about 500 μM. The dynamic range of the GPCR-based chemical biosensor or assay using the GPCR-based chemical biosensor can range from about 4 to about 68. The dynamic range is the ratio of the highest fluorescence obtained by the sensor in the presence vs. the absence of the chemical. It will be appreciated that the linear and dynamic range can be customized based on the configuration sensor unit, response unit, processing unit, and amplification unit, both individually and collectively as a system.

Sequences
Human 5-hydroxytryptamine Receptor 4(A)-
5-HTR_{4A} (Q13639-2)
(SEQ ID NO: 1)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLRY
TVLHRGHHQELEKLPIHNDPESLESCF.
Human 5-hydroxytryptamine Receptor 4(B)-
5-HTR_{4B} (Q13639-1)
(SEQ ID NO: 2)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLRD
AVECGGQWESQCHPPATSPLVAAQPSDT.
Human 5-hydroxytryptamine Receptor 4(C)-
5-HTR_{4C} (Q13639-9)
(SEQ ID NO: 3)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLSS
GTETDRKKLWNKEEKIDQTIQMPKRKRKKKASLSYEDLILLGRKS
CFREGK.
Human 5-hydroxytryptamine Receptor 4(D)-
5-HTR_{4D} (Q13639-3)
(SEQ ID NO: 4)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLRF.
Human 5-hydroxytryptamine Receptor 4(E)-
5-HTR_{4E} (X) S9
(SEQ ID NO: 5)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLSF
PLLFCNRPVPV.
Human 5-hydroxytryptamine Receptor 4(F)-
5-HTR_{4F} (X)
(SEQ ID NO: 6)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLSP
VPV.
Human 5-hydroxytryptamine Receptor 4(G)-
5-HTR_{4G} (Q13639-5)
(SEQ ID NO: 7)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLSG
CSPVSSFLLLFCNRPVPV.
Human 5-hydroxytryptamine Receptor 4(I)-
5-HTR_{4I} (Q13639-8)
(SEQ ID NO: 8)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLRT
DFLFDRDILARYWTKPARAGPFSGTLSIRCLTARKPVLGDAVECG
GQWESQCHPPATSPLVAAQPSDT.
Human 5-hydroxytryptamine Receptor 4(N)-
5-HTR_{4N} (Q13639-7)
(SEQ ID NO: 9)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLR.
In some embodiments, the GPCR has the amino
acid sequence
(SEQ ID NO: 10)
MDKLDANVSSEEGFGSVEKVVLLTFLSTVILMAILGNLLVMVAVC
WDRQLRKIKTNYFIVSLAFADLLVSVLVMPFGAIELVQDIWIYGE
VFCLVRTSLDVLLTTASIFHLCCISLDRYYAICCQPLVYRNKMTP
LRIALMLGGCWVIPTFISFLPIMQGWNNIGIIDLIEKRKFNQNSN
STYCVFMVNKPYAITCSVVAFYIPFLLMVLAYYRIYVTAKEHAHQ
IQMLQRAGASSESRPQSADQHSTHRMRTETKAAKTLCIIMGCFCL
CWAPFFVTNIVDPFIDYTVPGQVWTAFLWLGYINSGLNPFLYAFL
NKSFRRAFLIILCCDDERYRRPSILGQTVPCSTTTINGSTHVLX,

where X is any amino acid(s) from 1 to 100 amino acids in length, including 1-70, 1-80, 1-90, such as 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 amino acids in length.

Additional aspects of the present disclosure can be found, for example, in Marquez-Gomez P L, Damiano S R, Torp L R, Peralta-Yahya P. Modulating the Properties of GPCR-Based Sensors Via C-Terminus Isoforms. ACS Synth Biol. 2025 May 16; 14 (5): 1853-1860. doi: 10.1021/acssynbio.4c00847. Epub 2025 Apr. 25. PMID: 40279474; PMCID: PMC12090342 and the supplementary information of Marquez-Gomez P L, Damiano S R, Torp L R, Peralta-Yahya P. Modulating the Properties of GPCR-Based Sensors Via C-Terminus Isoforms. ACS Synth Biol. 2025 May 16; 14 (5): 1853-1860. doi: 10.1021/acssynbio.4c00847. Epub 2025 Apr. 25. PMID: 40279474; PMCID: PMC12090342, the entire contents of both of which are incorporated by reference as if fully set forth herein.

Additional aspects of GPCRs, including sequences, that can be utilized according to the present disclosure can be found, for example, in Langevin R, Martin-Downey M, Patel A, Archer H, Davila Severiano S J, Peralta-Yahya P. Tuning the Response of GPCR-Based Yeast Sensors Using Fluorescent Reporters. ACS Synth Biol. 2026 Jan. 16; 15 (1): 61-73. doi: 10.1021/acssynbio.5c00466. Epub 2025 Dec. 15. PMID: 41396999; PMCID: PMC12814776, the entire contents of which are incorporated by reference as if fully set forth herein, in particular, Supplementary Tables 7 and 8.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

In the present example, varying the GPCR C-terminus to rapidly modulate chemical biosensor properties is explored. Rather than using synthetic GPCR C-termini mutants and studying their ability to modulate sensor signal, natural GPCR C-terminus isoforms were used for this endeavor as it may shed light on their roles in different human tissue. Specifically, nine naturally occurring 5-HTR₄ C-terminus isoforms were screened to quickly optimize the dynamic and linear ranges of a serotonin sensor. To determine the extent to which the GPCR isoform-based sensor properties are ligand dependent, the nine sensors with two structurally dissimilar 5-HTR₄ agonists were evaluated. It is found that GPCR-based sensor properties are ligand dependent. Taken together, bioprospecting naturally occurring GPCR C-terminus isoforms is an effective strategy to rapidly optimize GPCR-based sensor properties.

### Results

Serotonin receptor 4 (5-HTR4) C-terminus isoforms

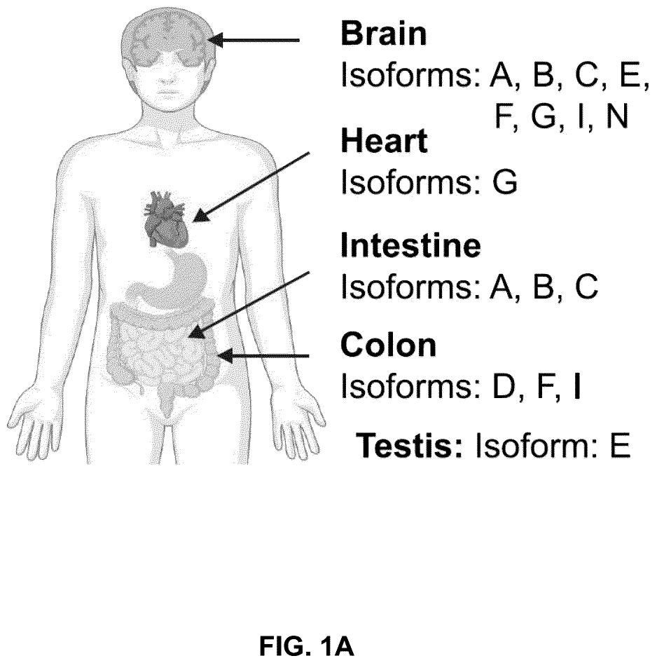

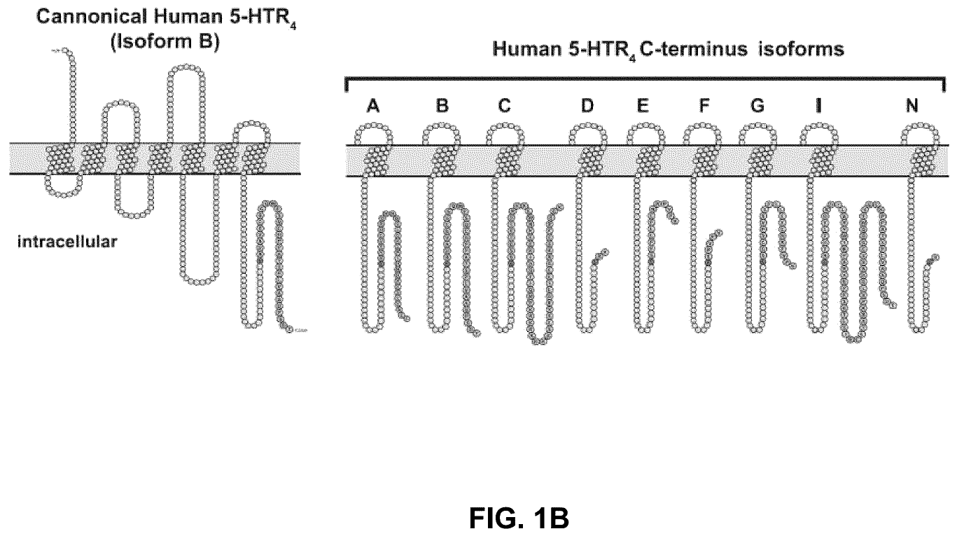

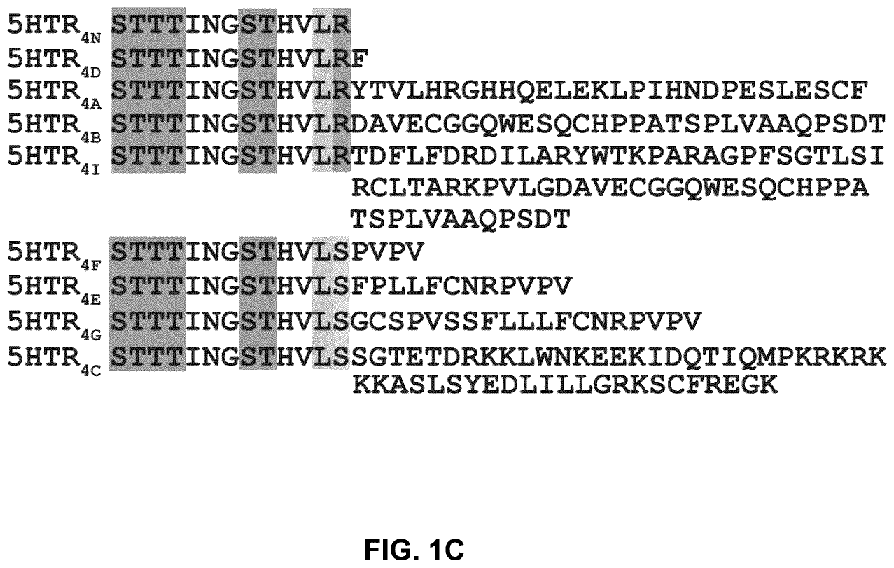

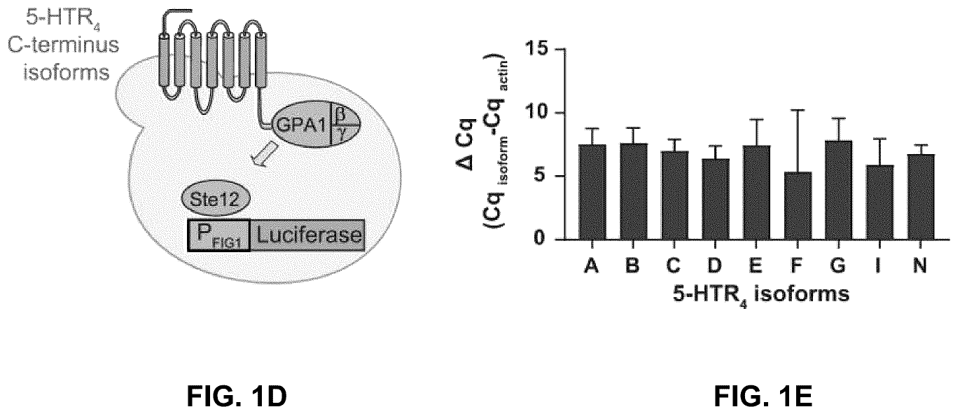

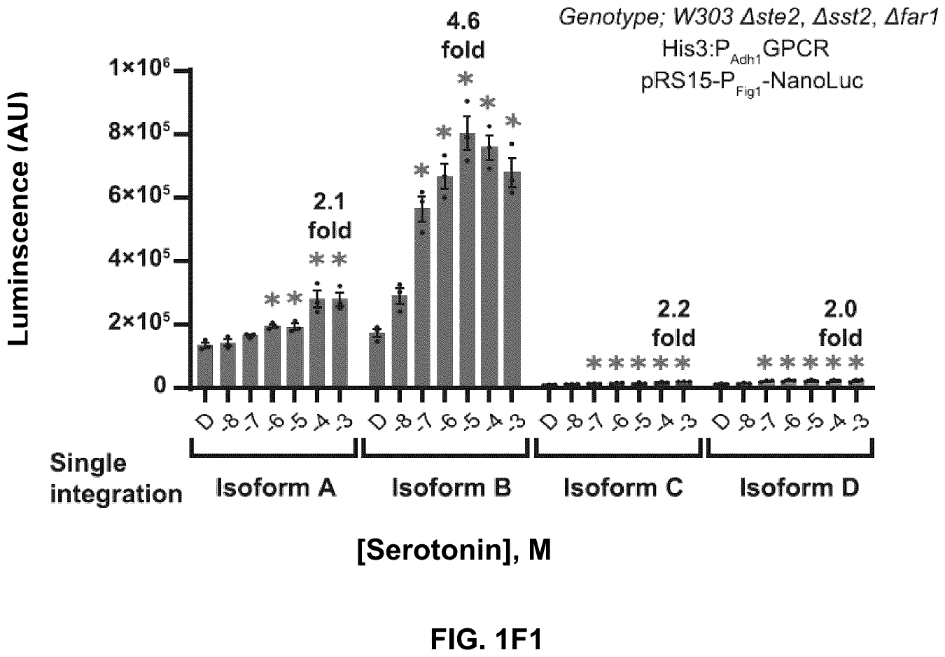

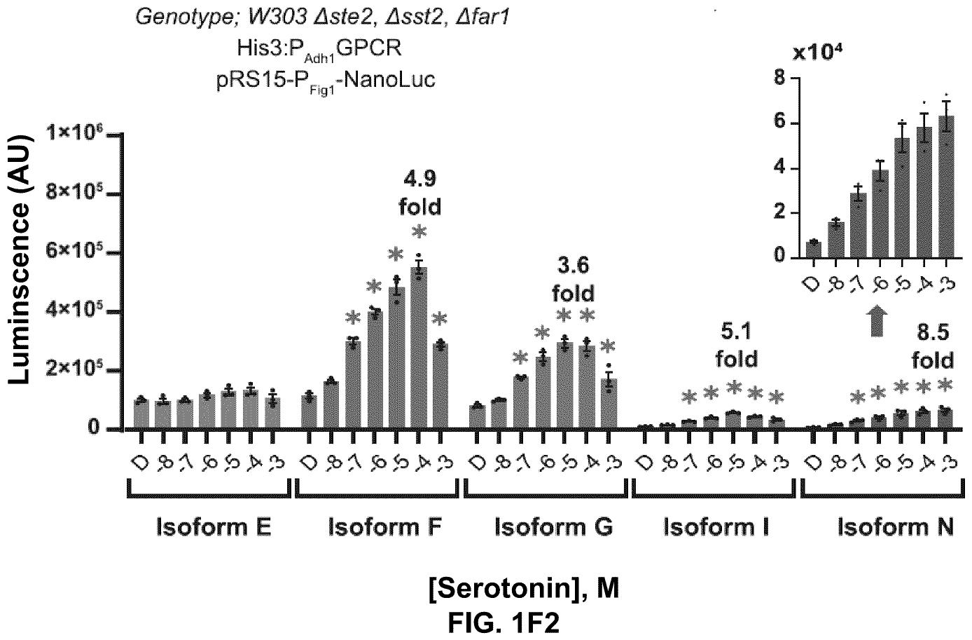

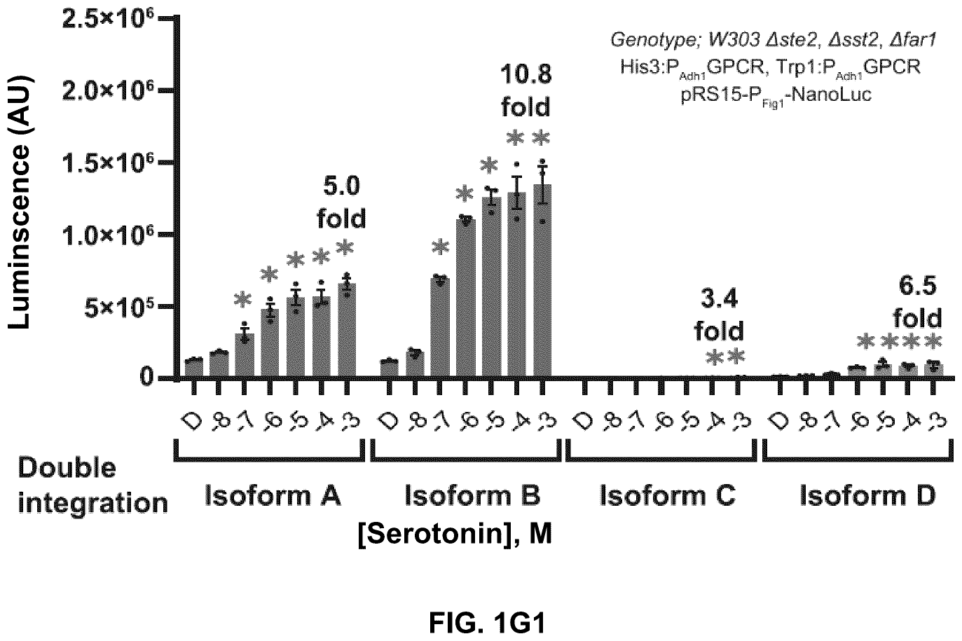

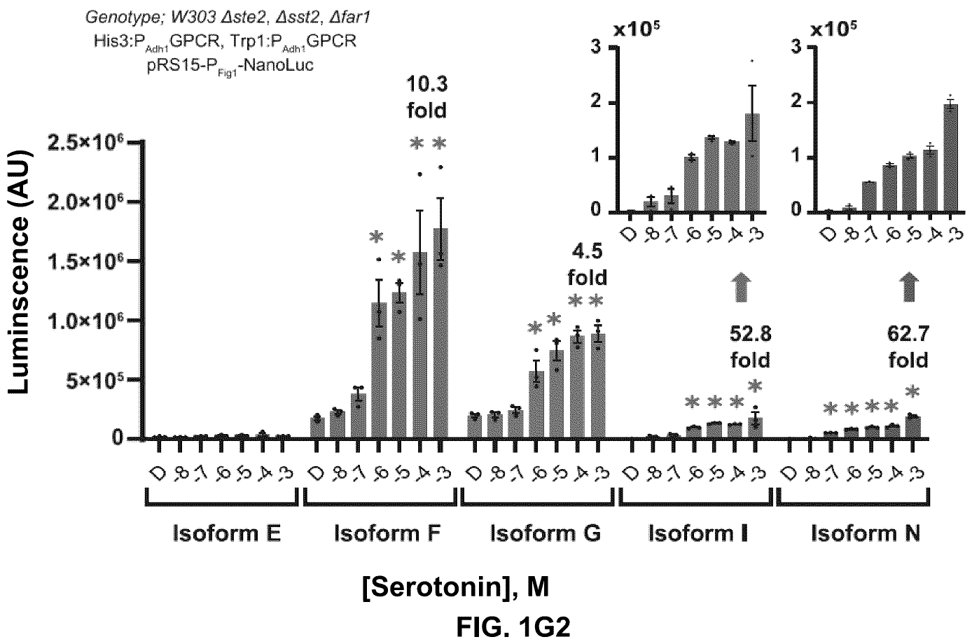

Nine of the ten functional 5-HTR₄ C-terminus isoforms have variations only at the C-terminus (Medhurst, A. D.; Lezoualc'h, F.; Fischmeister, R.; Middlemiss, D. N.; Sanger, G. J. Quantitative mRNA analysis of five C-terminal splice variants of the human 5-HT4 receptor in the central nervous system by TaqMan real time RT-PCR. Brain Res Mol Brain Res 2001, 90 (2), 125-134. DOI: 10.1016/s0169-328x (01) 00095-x)-5-HTR_{4A}, 5-HTR_{4B}, 5-HTR_{4C}, 5-HTR_{4D}, 5-HTR_{4E}, 5-HTR_{4F}, 5-HTR_{4G}, 5-HTR_{4I}, and 5-HTRAN-presenting themselves as the optimal set of receptors for this example. The 5-HTR₄ C-terminus isoforms have distinct expression in different tissue (Coupar, I. M.; Desmond, P. V.; Irving, H. R. Human 5-HT (4) and 5-HT (7) receptor splice variants: are they important? Curr Neuropharmacol 2007, 5 (4), 224-231. DOI: 10.2174/157015907782793621; Medhurst, A. D.; Lezoualc'h, F.; Fischmeister, R.; Middlemiss, D. N.; Sanger, G. J. Quantitative mRNA analysis of five C-terminal splice variants of the human 5-HT4 receptor in the central nervous system by TaqMan real time RT-PCR. Brain Res Mol Brain Res 2001, 90 (2), 125-134. DOI: 10.1016/s0169-328x (01) 00095-x; Oladosu, F. A.; Maixner, W.; Nackley, A. G., Alternative Splicing of G Protein-Coupled Receptors: Relevance to Pain Management. Mayo Clin Proc 2015, 90 (8), 1135-1151. DOI: 0.1016/j.mayocp.2015.06.010; Cartier, D.; Jegou, S.; Parmentier, F.; Lihrmann, I.; Louiset, E.; Kuhn, J. M.; Bastard, C.; Plouin, P. F.; Godin, M.; Vaudry, H.; Lefebvre, H. Expression profile of serotonin4 (5-HT4) receptors in adrenocortical aldosterone-producing adenomas. Eur J Endocrinol 2005, 153 (6), 939-947. DOI: 10.1530/eje.1.02051) with isoforms A and B expressed throughout the body, isoforms E, F, G, I and N found in the brain, isoform G found in the heart, isoform C found in the gastrointestinal tract, isoform D found in the colon and isoform E found in the testis (FIG. 1A). The nine 5-HTR₄ C-terminus isoforms share the same sequence up to L358, the last consensus amino acid (FIGS. 1B-1C, FIG. 3). The Cryo-EM structure of 5-HTR_{4B} ends at C329, thus no information can be gleaned about the secondary structure of the C-terminus23. AlphaFold models of the 5-HTR₄ C-terminus isoforms predict the C-terminus to be disordered except for 5-HTRc and 5-HTR_{4G}, in which the C-terminus forms a small intracellular helix (FIG. 3) (Jumper, J.; Evans, R.; Pritzel, A.; Green, T.; Figurnov, M.; Ronneberger, O.; Tunyasuvunakool, K.; Bates, R.; Zidek, A.; Potapenko, A.; Bridgland, A.; Meyer, C.; Kohl, S. A. A.; Ballard, A. J.; Cowie, A.; Romera-Paredes, B.; Nikolov, S.; Jain, R.; Adler, J.; Back, T.; Petersen, S.; Reiman, D.; Clancy, E.; Zielinski, M.; Steinegger, M.; Pacholska, M.; Berghammer, T.; Bodenstein, S.; Silver, D.; Vinyals, O.; Senior, A. W.; Kavukcuoglu, K.; Kohli, P.; Hassabis, D., Highly accurate protein structure prediction with AlphaFold. Nature 2021, 596 (7873), 583-589. DOI: 10.1038/s41586-021-03819-2). FIG. 1D shows a schematic of 5-HTR₄ C-terminus isoform-based sensor in yeast. Human 5-HTR₄ isoform (blue) is expressed on the yeast cell surface

### 5-HTR₄ C-Terminus Isoform-Based Sensor Construction

Previously, a yeast 5-HTR_{4B}-based sensor was engineered by coupling 5-HTR_{4B} activation to the yeast Ga subunit, GPA1, ultimately resulting in cell luminescence (Yasi, E. A.; Allen, A. A.; Sugianto, W.; Peralta-Yahya, P. Identification of Three Antimicrobials Activating Serotonin Receptor 4 in Colon Cells. ACS Synth Biol 2019, 8 (12), 2710-2717. DOI: 10.1021/acssynbio.9b00310.). The human 5-HTR_{4B} was expressed from a strong promoter (P_{Tef1}) using a multi-copy plasmid (pESC), while the reporter gene was controlled by the mating pathway promoter Fig₁ (P_{Fig1}) using a single copy plasmid. Although this system resulted in up to 32-fold increase in signal after activation in the presence of serotonin, the biosensor was noisy due expression of the GPCR from a multi-copy plasmid. In this work, the biosensor signal noise was reduced by integrating 5-HTR_{4B}, 5-HTR_{4A}, 5-HTR_{4C}, 5-HTR_{4D}, 5-HTR_{4E}, 5-HTR_{4F}, 5-HTR_{4G}, 5-HTR_{4I}, or 5-HTRAN into the yeast genome of the GPCR biosensor strain (W303 Δste2, Δsst2, Δfar1²⁵). Briefly, the 5-HTR₄ C-terminus isoforms were placed under control of the medium strength promoter P_{ADH1} and integrated at the His3 locus. The luminescence reporter gene was kept under control of P_{Fig1} as a single copy plasmid. After genome integration of the 5-HTR₄ C-terminus isoforms, the isoforms were confirmed to be similarly expressed by performing real time-PCR. As shown in FIG. 1E, the ΔCq for the isoform are not statistically different from one another.

Single Integrated 5-HTR₄ C-Terminus Isoform-Based Sensor Characterization with Serotonin.

Each 5-HTR₄ C-terminus isoform has a unique profile for signal after activation in the presence of serotonin (FIGS. 1F1-1F2). Each 5-HTR₄ C-terminus isoform has a unique profile for signal after activation in the presence of serotonin (FIGS. 1F1-1F2). Isoform B, the canonical 5-HTR₄ isoform, has a linear range from 10⁻⁷-10⁻⁵ M achieving up to a 4.6-fold increase in signal after activation. Although Isoform B achieves the highest raw luminescence, it has a high basal sensor activity, i.e. activity in the presence of the carrier solvent, which reduces the overall dynamic range of the sensor. Isoform N, the isoform with the shortest C-terminus, has the lowest basal sensor activity, with a linear range spanning five orders of magnitude from 10⁻⁷-10⁻³ M serotonin and achieving up to 8.5-fold increase in signal after activation. The different linear ranges achieved with Isoform B (10⁻⁷-10⁻⁵ M) and Isoforms N (10⁻⁷-10⁻³ M) make them valuable for different applications. For example, if the objective is to detect serotonin in the 10⁻⁶ M range, the Isoform B-based sensor is preferred as it has a linear behavior in that range. However, if the objective is to detect serotonin in the 10⁻⁴ M range, the Isoform N-based sensor is better suited for that application as Isoform N has a linear behavior in that range. Isoforms A, C and D perform similarly, achieving on average 2.1-fold increases in signal after activation at 10⁻⁴-10⁻³ M serotonin. Interestingly, Isoform E is not activated by serotonin at any concentration. A last isoform worth highlighting is Isoform F, which has a 10⁻⁷-10⁻⁴ linear range and achieves a 4.9-fold increase in signal after activation at 10⁻⁴ M serotonin.

### Double Integrated 5-HTR₄ C-Terminus Isoform-Based Sensor Construction and Characterization.

In the context of yeast GPCR-based sensors, maximum signal increase after activation has been observed when two copies of the GPCR are integrated in the genome (Rowe, J. B.; Taghon, G. J.; Kapolka, N. J.; Morgan, W. M.; Isom, D. G., CRISPR-addressable yeast strains with applications in human G protein-coupled receptor profiling and synthetic biology. J Biol Chem 2020, 295 (24), 8262-8271. DOI: 10.1074/jbc.RA120.013066). Interested in determining if a second integration of the 5-HTR₄ C-terminus isoforms would increase the sensors' signal after activation, a second copy of each isoform was introduced also under control of the PADH1 at the Trp1 locus. As shown in FIGS. 1G1-1G2, the signal after activation of all doubly integrated 5-HTR₄ C-terminus isoform-based sensors increases. Double integration of isoform B or Isoform F results in a ~10-fold increase in signal after activation. Most remarkable, however, is the performance of Isoform I and Isoform N that jump to 52.8-fold and 62.7-fold increases in signal after activation, respectively. Pivotal to the sensor performance improvement seen for Isoforms I and N is their maintenance of a very low basal sensor activation.

Characterization of 5-HTR₄ C-Terminus Isoform-Based Sensors with Known Serotonin Agonists.

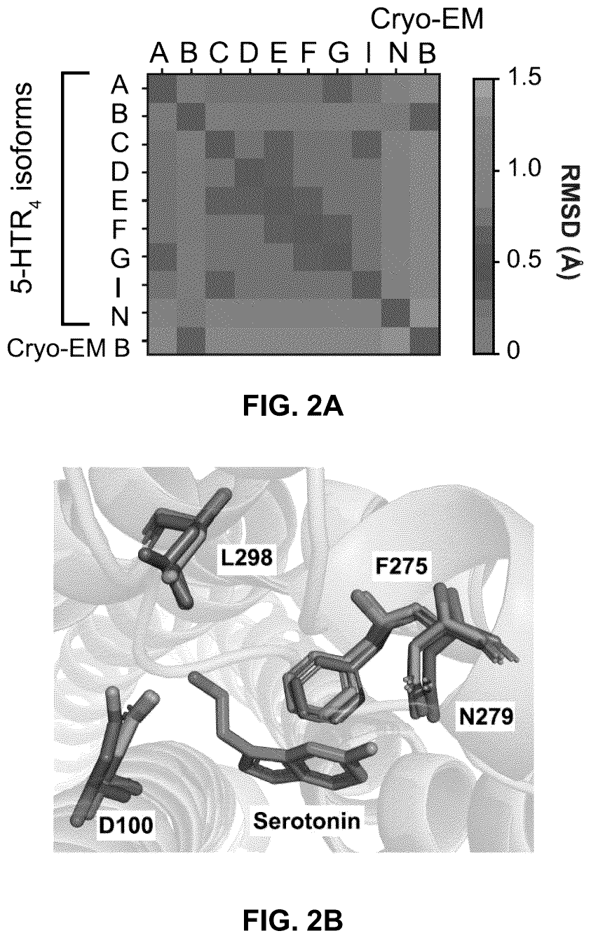

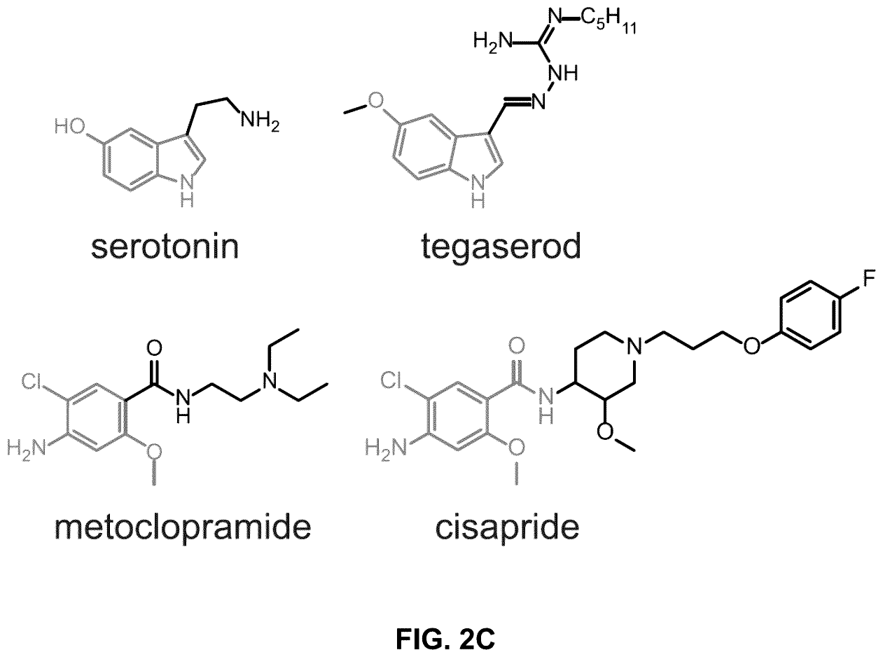

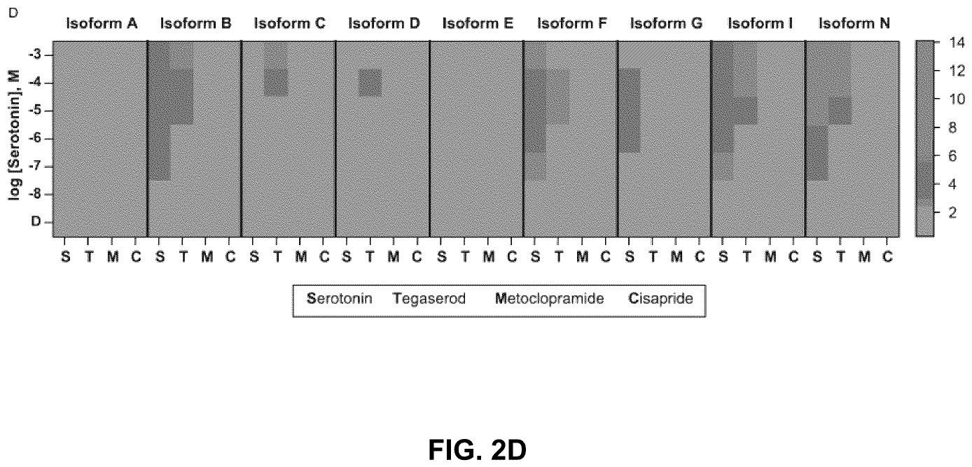

Given the nine 5-HTR₄ C-terminus isoforms expressed at similar levels (FIG. 1E), and that the chemical binding site of the isoforms are almost identical (FIGS. 2A-2B)-<1 Å RMSD across the active sites of the AlphaFold modeled isoforms—it was thought that the system could be used to evaluate how structurally diverse 5-HTR₄ ligands (FIG. 2C) affect GPA1 coupling and ultimate sensor signaling. It is well established that GPCR signaling response can be modulated by the ligand itself, which induces rearrangement of the transmembrane helices (vHilger, D.; Masureel, M.; Kobilka, B. K. Structure and dynamics of GPCR signaling complexes. Nat Struct Mol Biol 2018, 25 (1), 4-12. DOI: 10.1038/s41594-017-0011-7) resulting in different positions of the intracellular loops and C-terminus interacting with the G-protein, ultimately affecting signaling. For example, tegaserod acts as a 5-HTR₄ agonist while piboserod acts as an antagonist. In the context of 5-HTR₄ C-terminus isoforms, there is evidence that they respond differently to the same ligand. In mammalian CHO cells, renzapride is almost 20 times more potent at activating 5-HTR_{4D} than 5-HTR_{4G} measured as CAMP accumulation²⁹.

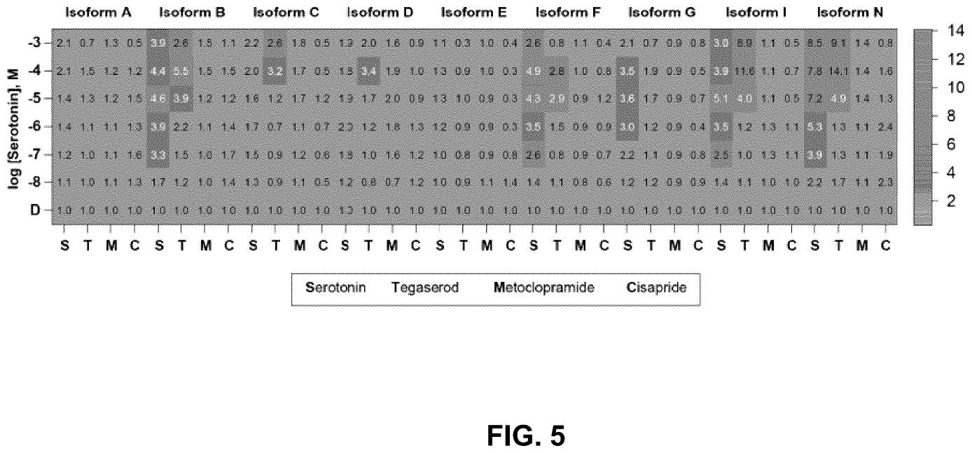

The properties of 5-HTR₄ C-terminus isoform-based sensors with three structurally different agonists were evaluated: tegaserod, metoclopramide and cisapride (FIG. 2C). Structurally, tegaserod shares the 5-hydroxyindole moiety with serotonin, while metoclopramide and cisapride use a hydroxyaniline moiety to provide the necessary hydrophobicity. FIG. 2D (FIGS. 4A-4I, FIG. 5) shows the x-fold increase in signal after activation of the nine 5-HTR4 C-terminus isoform-based sensors with serotonin, tegaserod, metoclopramide and cisapride. It was observed that the sensors result in different fold activations when activated by different compounds despite having an almost identical active site. For example, the canonical Isoform B is activated by both serotonin (4.6-fold) and tegaserod (5.5-fold) which share the 5-hydroxyindole moiety, but not by metoclopramide or cisapride that share a hydroxyaniline moiety. Interestingly, Isoform I is more strongly activated by tegaserod (11.6-fold) than serotonin (5.6-fold).

### CONCLUSION

In this work, the utility of GPCR C-terminus isoforms to rapidly modulate the properties of a chemical biosensor was evaluated. It was found that bioprospecting naturally occurring GPCR-based C-terminus isoforms can be a successful strategy in expanding both the linear and dynamic ranges of GPCR-based sensors. Taken together, serotonin sensors spanning 2-8.5-fold increases in signal after activation as well as ranging over 5 orders of magnitude −10⁻⁷-10⁻³ M serotonin—were generated. This suite of 5-HTR₄ C-terminus isoform-based sensor now allows picking serotonin sensors for different applications. For example, if detection of 10⁻⁶ M serotonin is desired, the Isoform B-based sensor would be the best choice as it has a linear range around that concentration. On the other hand, if detection of 10⁻⁴M serotonin is desired, Isoform N-based sensor would be the preferred one as it has a linear range around that concentration. It was also found that the double integration of 5-HTR₄-based sensors in the chromosome significantly increase sensor signal over that seen in the single integrated sensor versions. The low basal activity of Isoform I and Isoform N allows them to achieve more than a 50-fold increase in signal after activation.

Interestingly, GPCR isoform-based sensors have different properties depending on the ligands used. The analysis performed with three known 5-HTR₄ agonists, tegaserod, metoclopramide and cisapride, shows how isoforms are differentially activated by these ligands hinting at GPCR isoforms potentially being differentially activated throughout the body by different ligands.

Placing this work in the broader biosensor context, unlike allosteric transcription factor-based sensors in which the sensor's features can only be modulated by engineering the ligand binding domain or altering the DNA operator sequence (Patel, A.; Peralta-Yahya, P. Olfactory Receptors as an Emerging Chemical Sensing Scaffold. Biochemistry 2023, 62 ( )2), 187-195. DOI: 10.1021/acs.biochem.2c00486, GPCR-based sensors can be additionally modulated by changing their GPCR C-terminus. Improved coupling of human GPCR to the yeast machinery likely increases the active concentration of the mating pathway transcription factor Ste12, resulting in higher reporter gene transcription. Finally, changes to the GPCR C-terminus should work synergistically with changes to other parts of the system towards enhancing the sensor's properties, including improving the GPCR-ligand interactions and the transcription factor/promoter affinity.

### Materials and Methods

Materials. Serotonin hydrochloride (S0370) was purchased from TCI chemicals. Metoclopramide hydrochloride (M0763) and Tegaserod maleate (SML1504) were purchased from Sigma-Aldrich. Nano-Glo® Luciferase Assay System (N1120) was purchased from Promega.

5-HTR4 C-terminus isoforms plasmid construction (integration plasmids). T For the single integrated 5-HTR₄ C-terminus isoform-based sensors, the 5-HTR₄ C-terminus isoforms were amplified from their respective pESC vectors using primers that introduced a FLAG tag at the N-terminus. The FLAG-tagged 5-HTR₄ C-terminus isoforms were introduced into pNH603-C. glabrata His3-P_{ADH1}-MCP-VP64-C. albicans T_{ADH1} (pJZC522³⁰) between NotI/XhoI (replacing MCP-VP64) via Gibson assembly to generate pJZC522-P_{ADH1}-FLAG-5-HTR_{4A} (pPM136), pJZC522-P_{ADH1}-FLAG-5-HTR_{4B} (pPM137), pJZC522-P_{ADH1}-FLAG-5-HTR_{4C} (pPM138), pJZC522-P_{ADH1}-FLAG-5-HTR_{4D} (pPM139), pJZC522-P_{ADH1}-FLAG-5-HTR_{4E} (pPM140), pJZC522-P_{ADH1}-FLAG-5-HTR_{4F} (pPM141), pJZC522-P_{ADH1}-FLAG-5-HTR_{4G} (pPM142), pJZC522-P_{ADH1}-FLAG-5-HTR_{4I} (pPM143), and pJZC522-P_{ADH1}-FLAG-5-HTR_{4N} (pPM144). Constructs were sequence verified using whole plasmid sequencing. To generate the integration plasmids for the double integrated 5-HTR₄ C-terminus isoform-based sensors, the P_{ADH1}-FLAG-5-HTR₄ C-terminus isoforms were amplified from their respective pJZC522 vectors and introduced into pJZC530-C. glabrata Trp1-P_{TEF1}-GPA1-5AA-Gₛ-CA T_{ADH1} (PPM189) between ApaI/BamHI (replacing P_{TEF1}-GPA1-5AA-Gₛ) via Gibson assembly to generate pPM189-P_{ADH1}-FLAG-5-HTR_{4A} (pPM289), pPM189-P_{ADH1}-FLAG-5-HTR_{4B} (pPM290), pPM189-P_{ADH1}-FLAG-5-HTR_{4C} (pPM291), pPM189-P_{ADH1}-FLAG-5-HTR_{4D} (pPM292), pPM189-P_{ADH1}-FLAG-5-HTR_{4E} (pPM293), pPM189-P_{ADH1}-FLAG-5-HTR_{4F} (pPM294), pPM189-P_{ADH1}-FLAG-5-HTR_{4G} (pPM295), pPM189-P_{ADH1}-FLAG-5-HTR_{4I} (pPM296), and pPM189-P_{ADH1}-FLAG-5-HTR_{4N} (pPM297). Constructs were sequence verified using whole plasmid sequencing.

Single integrated 5-HTR₄ C-terminus isoform-based sensor strain construction pPM136, pPM137, pPM138, pPM139, pPM140, pPM141, pPM142, pPM143, and pPM144 were linearized by digesting with Pme/and transforming the linear DNA into PPY140 (S. cerevisiae W303 leu2-3,112 trp1-1 can1-100 ura3-1 ade2-1 his3-11,15 Δfar1, Δste2, Δsst2)²⁵. Successful yeast integrations were selected on media lacking histidine. GPCR integrations were verified via PCR using primers PB140/PB141. The single integrated 5-HTR₄ C-terminus strains PPY2753 (5-HTR_{4A}), PPY2754 (5-HTR_{4B}), PPY2755 (5-HTR_{4C}), PPY2756 (5-HTR_{4D}), PPY2763 (5-HTR_{4E}), PPY2757 (5-HTR_{4F}), PPY2758 (5-HTR_{4G}), PPY2759 (5-HTR_{4I}), and PPY2760 (5-HTR_{4N}) were transformed with pRS415-Leu2-P_{FIG1}-NanoLuc²² to generate sensors for 5-HTR_{4A} (PPY2796), 5-HTR_{4B} (PPY2797), 5-HTR_{4C} (PPY2798), 5-HTR_{4D} (PPY2799), 5-HTR_{4E} (PPY2800), 5-HTR_{4F} (PPY2801), 5-HTR_{4G} (PPY2802), 5-HTR_{4I} (PPY2803), and 5-HTR_{4N} (PPY2804). The no receptor control (PPY2805), i.e. the GPCR-based sensors strain carrying no integrated receptor was generated by transforming pRS415-Leu2-P_{FIG1}-NanoLuc²² into PPY140.

Double integrated 5-HTR₄ C-terminus isoform-based sensor strain construction pPM289, pPM290, pPM291, pPM292, pPM293, pPM294, pPM295, pPM296, and pPM297 were linearized by digesting with Pmel and transforming the linear DNA into PPY2753 (5-HTR_{4A}), PPY2754 (5-HTR_{4B}), PPY2755 (5-HTR_{4C}), PPY2756 (5-HTR_{4D}), PPY2763 (5-HTR_{4E}), PPY2757 (5-HTR_{4F}), PPY2758 (5-HTR_{4G}), PPY2759 (5-HTR_{4I}), and PPY2760 (5-HTR_{4N}). Successful yeast integrations were selected on media lacking histidine and tryptophan. GPCR integrations were verified via PCR using primers PB140/PB142. PPY3533 (5-HTR_{4A}), PPY3534 (5-HTR_{4B}), PPY3535 (5-HTR_{4C}), PPY3536 (5-HTR_{4D}), PPY3537 (5-HTR_{4E}), PPY3538 (5-HTR_{4F}), PPY3539 (5-HTR_{4G}), PPY3540 (5-HTR_{4I}), and PPY3541 (5-HTR_{4N}) were transformed with pRS415-Leu2-P_{FIG1}-NanoLuc to generate sensors for 5-HTR_{4A} (PPY3598), 5-HTR_{4B} (PPY3599), 5-HTR_{4C} (PPY3600), 5-HTR_{4D} (PPY3601), 5-HTR_{4E} (PPY3602), 5-HTR_{4F} (PPY3603), 5-HTR_{4G} (PPY3604), 5-HTR_{4I} (PPY3605), and 5-HTR_{4N} (PPY3606).

5-HTR₄ C-terminus isoform-based sensor activation. Single integrated 5-HTR₄ C-terminus isoform-based sensors. Overnight cultures for three independent colonies of each PPY2796, PPY2797, PPY2798, PPY2799, PPY2800, PPY2801, PPY2802, PPY2803, or PPY2804 were used to inoculate 50 mL of synthetic complete medium with 2% glucose lacking histidine and leucine (SD(HL⁻)) to an OD₆₀₀=0.06. After 18 h at 15° C. (150 rpm), the cultures were centrifuged (3500 rpm, 10 min), and resuspended in SD (HL⁻) to an OD₆₀₀=1. In a white, flat-bottomed 96-well plate, 190 μl pH=7 SD (HL⁻), 8 μl of cells, and 2 μl of serotonin (final concentration 10⁻⁹-10⁻³ M), or DMSO as a control were added. After chemical incubation (2.5 h, 30° C., 250 rpm), 20 μl of 1:100 mixture of NanoLuc substrate to NanoLuc buffer were added, and the reaction incubated for 30 min (30° C., 250 rpm). Luminescence was read in a Biotek Synergy 2 using default settings. The same protocol was followed to detect tegaserod, metoclopramide and cisapride. Double integrated 5-HTR₄ C-terminus isoform-based sensors. Overnight cultures for three independent colonies of each PPY3598, PPY3599, PPY3600, PPY3601, PPY3602, PPY3603, PPY3604, PPY3605, or PPY3606 were used to inoculate 5 mL of synthetic complete medium with 2% glucose lacking histidine, tryptophan and leucine (SD(HWL⁻)) to an OD₆₀₀=0.6. After 18 h at 15° C. (150 rpm), the cultures were centrifuged (3500 rpm, 10 min), and resuspended in SD(HWL⁻) to an OD₆₀₀=1. In a white, flat-bottomed 96-well plate, 190 μl pH=7 SD (HWL⁻), 8 μl of cells, and 2 μl of serotonin (final concentration 10⁻⁹-10⁻³ M), or DMSO as a control were added. After chemical incubation (2.5 h, 30° C., 250 rpm), 20 μl of 1:100 mixture of NanoLuc substrate to NanoLuc buffer were added, and the reaction incubated for 30 min (30° C., 250 rpm). Luminescence was read in a Biotek Synergy 2 using default settings.

mRNA quantification. Overnight cultures for three independent colonies of PPY2796, PPY2797, PPY2798, PPY2799, PPY2800, PPY2801, PPY2802, PPY2803, or PPY2804 were used to inoculate 50 mL SD (HL⁻) to an OD 600=0.06. After 18 h at 15° C. (150 rpm), the cultures were centrifuged (3500 rpm, 10 min), and resuspended in SD (HL−) to an OD₆₀₀=1. 1 mL of cells at OD=1 were pelleted, and total RNA was extracted using RNeasy Mini kit (Qiagen). RNA concentrations were measured using a Nanodrop Lite spectrophotometer. Reverse transcription was done using 1000 ng of total RNA using QuantiTect Reverse Transcription kit (Qiagen). Real time PCR reactions were set up using the QuantiTect SYBR Green PCR kit (Qiagen) using 3 μL of 250 ng of cDNA and read using an Applied Biosciences StepOnePlus Real-Time PCR system. Reactions were set up as triplicates using primers PM76/PM77 for all isoforms and ACT-F/ACT-R for actin. 5-HTR₄ C-terminus isoform expression was normalized to the housekeeping gene, ACT1 and were compared using comparative Cq method using the equations below:

$$Δ {C}_{q} = avg {C}_{q \_ sample} - {avgC}_{q \_ Actin} \qquad ( eq . 1 )$$

AlphaFold isoform structures. The structure of 5-HTR_{4B} structure was obtained from the AlphaFold web server (Q13639) 24. The structures of 5-HTR₄ isoforms A, C, D, E, F, G, I and N were obtained using the monomer model in AlphaFold Colab

RMSD calculations. The cryo-EM structure, 5-HTR_{4B}-Gs complex (PDB: 7XT9), and AlphaFold structures of 5-HTR₄: A, B, C, D, E, F, G, I, N were entered into PyMOL. To determine the binding pocket, residues 5 Å from serotonin in the cryo-EM structure and AlphaFold structures were selected. PyMOL was used to obtain the RMSD value for the binding pocket.

TABLE 1
Table of Plasmids

| Plasmid | Plasmid | | |
|---|---|---|---|
| Number | Name | Description | Citation |
| PPY111 | pKM111 | pESC-His3-P_{TEF} | 1 |
| PPY1192 | pTMC18 | pESC-His3-P_{TEF1}-5-HTR_{4B} | 2 |
| PPY1740 | pEY15 | pRS415-Leu2-P_{FIG1}-NanoLuc | 3 |
| PPY2161 | pJZC522 | pNH603-C. glabrata His3-P_{ADH1}-MCP-VP64-C. albicans | 4 |
| | | T_{ADH1} | |
| PPY2162 | pJZC530 | pNH604-C. glabrata Trp1-P_{TetO1x}-Venus- C. albicans T_{ADH1} | 5 |
| PPY2084 | pLT3 | pESC-HIS3-P_{TEF1}-5-HTR_{4I} | This |
| | | | work |
| PPY2085 | pLT4 | pESC-HIS3-P_{TEF1}-5-HTR_{4N} | This |
| | | | work |
| PPY2378 | pPM58 | pESC-His3-P_{TEF1}-5-HTR_{4A} | This |
| | | | work |
| PPY2380 | pPM59 | pESC-His3-P_{TEF1}-5-HTR_{4G} | This |
| | | | work |
| PPY2381 | pPM61 | pESC-His3-P_{TEF1}-5-HTR_{4E} | This |
| | | | work |
| PPY2382 | pPM62 | pESC-His3-P_{TEF1}-5-HTR_{4C} | This |
| | | | work |
| PPY2383 | pPM63 | pESC-His3-P_{TEF1}-5-HTR_{4D} | This |
| | | | work |
| PPY2395 | pPM60 | pESC-His3-P_{TEF1}-5-HTR_{4F} | This |
| | | | work |
| PPY2734 | pPM136 | pInt-His-P_{ADH1}-FLAG-5-HTR_{4A}-CA T_{ADH1} | This |
| | | | work |
| PPY2735 | pPM137 | pInt-His-P_{ADH1}-FLAG-5-HTR_{4B}-CA T_{ADH1} | This |
| | | | work |
| PPY2736 | pPM138 | pInt-His-P_{ADH1}-FLAG-5-HTR_{4C}-CA T_{ADH1} | This |
| | | | work |
| PPY2737 | pPM139 | pInt-His-P_{ADH1}- FLAG-5-HTR_{4D}-CA T_{ADH1} | This |
| | | | work |
| PPY2738 | pPM140 | pInt-His-P_{ADH1}-FLAG-5-HTR_{4E}-CA T_{ADH1} | This |
| | | | work |
| PPY2739 | pPM141 | pInt-His-P_{ADH1}-FLAG-5-HTR_{4F}-CA T_{ADH1} | This |
| | | | work |
| PPY2740 | pPM142 | pInt-His-P_{ADH1}-FLAG-5-HTR_{4G}-CA T_{ADH1} | This |
| | | | work |
| PPY2741 | pPM143 | pInt-His-P_{ADH1}-FLAG-5-HTR_{4I}-CA T_{ADH1} | This |
| | | | work |
| PPY2742 | pPM144 | pInt-His-P_{ADH1}-FLAG-5-HTR_{4N}-CA T_{ADH1} | This |
| | | | work |
| PPY2937 | pPM189 | pInt-Trp-P_{TEF1}-GPA1-5AA-Gₛ-CA T_{ADH1} | This |
| | | | work |
| PPY3480 | pPM289 | pInt-Trp-P_{ADH1}-FLAG-5-HTR_{4A}-CA T_{ADH1} | This |
| | | | work |
| PPY3481 | pPM290 | pInt-Trp-P_{ADH1}-FLAG-5-HTR_{4B}-CA T_{ADH1} | This |
| | | | work |
| PPY3482 | pPM291 | pInt-Trp-P_{ADH1}-FLAG-5-HTR_{4C}-CA T_{ADH1} | This |
| | | | work |
| PPY3483 | pPM292 | pInt-Trp-P_{ADH1}-FLAG-5-HTR_{4D}-CA T_{ADH1} | This |
| | | | work |
| PPY3484 | pPM293 | pInt-Trp-P_{ADH1}-FLAG-5-HTR_{4E}-CA T_{ADH1} | This |
| | | | work |
| PPY3485 | pPM294 | pInt-Trp-P_{ADH1}-FLAG-5-HTR_{4F}-CA T_{ADH1} | This |
| | | | work |
| PPY3486 | pPM295 | pInt-Trp-P_{ADH1}-FLAG-5-HTR_{4G}-CA T_{ADH1} | This |
| | | | work |
| PPY3487 | pPM296 | pInt-Trp-P_{ADH1}-FLAG-5-HTR_{4I}-CA T_{ADH1} | This |
| | | | work |
| PPY3488 | pPM297 | pInt-Trp-P_{ADH1}-FLAG-5-HTR_{4N}-CA T_{ADH1} | This |

TABLE 2
Table of Strains

| Strain Number | Description | Citation |
|---|---|---|
| PPY140 | S. cerevisiae W303MATa ade2-1 ura3-1 his3-11 trp1-1 | ATCC |
| | leu2-3 leu2-112 can1-100 Δfar1 Δste2 Δsst2 | |
| PPY2753 | PPY140 His3:P_{ADH1}-FLAG-5-HTR_{4A}-CA T_{ADH1} | This work |
| PPY2754 | PPY140 His3:P_{ADH1}-FLAG-5-HTR_{4B}-CA T_{ADH1} | This work |
| PPY2755 | PPY140 His3:P_{ADH1}-FLAG-5-HTR_{4C}-CA T_{ADH1} | This work |
| PPY2756 | PPY140 His3:P_{ADH1}-FLAG-5-HTR_{4D}-CA T_{ADH1} | This work |
| PPY2763 | PPY140 His3:P_{ADH1}-FLAG-5-HTR_{4E}-CA T_{ADH1} | This work |
| PPY2757 | PPY140 His3:P_{ADH1}-FLAG-5-HTR_{4F}-CA T_{ADH1} | This work |
| PPY2758 | PPY140 His3:P_{ADH1}-FLAG-5-HTR_{4G}-CA T_{ADH1} | This work |
| PPY2759 | PPY140 His3:P_{ADH1}-FLAG-5-HTR_{4I}-CA T_{ADH1} | This work |
| PPY2760 | PPY140 His3:P_{ADH1}-FLAG-5-HTR_{4N}-CA T_{ADH1} | This work |
| PPY2796 | PPY2753, pRS415-Leu2-P_{FIG1}-NanoLuc | This work |
| PPY2797 | PPY2754, pRS415-Leu2-P_{FIG1}-NanoLuc | This work |
| PPY2798 | PPY2755, pRS415-Leu2-P_{FIG1}-NanoLuc | This work |
| PPY2799 | PPY2756, pRS415-Leu2-P_{FIG1}-NanoLuc | This work |
| PPY2800 | PPY2763, pRS415-Leu2-P_{FIG1}-NanoLuc | This work |
| PPY2801 | PPY2757, pRS415-Leu2-P_{FIG1}-NanoLuc | This work |
| PPY2802 | PPY2758, pRS415-Leu2-P_{FIG1}-NanoLuc | This work |
| PPY2803 | PPY2759, pRS415-Leu2-P_{FIG1}-NanoLuc | This work |
| PPY2804 | PPY2760, pRS415-Leu2-P_{FIG1}-NanoLuc | This work |
| PPY2805 | PPY140, pRS415-Leu2-P_{FIG1}-NanoLuc | This work |
| PPY3533 | PPY2753 Trp1:P_{ADH1}-FLAG-5-HTR_{4A}-CA T_{ADH1} | This work |
| PPY3534 | PPY2754 Trp1:P_{ADH1}-FLAG-5-HTR_{4B}-CA T_{ADH1} | This work |
| PPY3535 | PPY2755 Trp1:P_{ADH1}-FLAG-5-HTR_{4C}-CA T_{ADH1} | This work |
| PPY3536 | PPY2756 Trp1:P_{ADH1}-FLAG-5-HTR_{4D}-CA T_{ADH1} | This work |
| PPY3537 | PPY2763 Trp1:P_{ADH1}-FLAG-5-HTR_{4E}-CA T_{ADH1} | This work |
| PPY3538 | PPY2757 Trp1:P_{ADH1}-FLAG-5-HTR_{4F}-CA T_{ADH1} | This work |
| PPY3539 | PPY2758 Trp1:P_{ADH1}-FLAG-5-HTR_{4G}-CA T_{ADH1} | This work |
| PPY3540 | PPY2759 Trp1:P_{ADH1}-FLAG-5-HTR_{4I}-CA T_{ADH1} | This work |
| PPY3541 | PPY2760 Trp1:P_{ADH1}-FLAG-5-HTR_{4N}-CA T_{ADH1} | This work |
| PPY3598 | PPY3533, pRS415-Leu2-P_{FIG1}-NanoLuc | This work |
| PPY3599 | PPY3534, pRS415-Leu2-P_{FIG1}-NanoLuc | This work |
| PPY3600 | PPY3535, pRS415-Leu2-P_{FIG1}-NanoLuc | This work |
| PPY3601 | PPY3536, pRS415-Leu2-P_{FIG1}-NanoLuc | This work |
| PPY3602 | PPY3537, pRS415-Leu2-P_{FIG1}-NanoLuc | This work |
| PPY3603 | PPY3538, pRS415-Leu2-P_{FIG1}-NanoLuc | This work |
| PPY3604 | PPY3539, pRS415-Leu2-P_{FIG1}-NanoLuc | This work |
| PPY3605 | PPY3540, pRS415-Leu2-P_{FIG1}-NanoLuc | This work |
| PPY3606 | PPY3541, pRS415-Leu2-P_{FIG1}-NanoLuc | This work |

TABLE 3
Table of Primers

| Primer | |
|---|---|
| name | Sequence |
| PM86 | tcaactatctcatatacaatctctctcgagATGGACTACAAAGACGACGACGACAAAGGCT |
| (SEQ ID | CAGGCgataagttggatgctaacgtttct |
| NO: 11) | |
| PM87 | gcttagagctccaccgcggtggcggccgctcagaagcatgattccaggga |
| (SEQ ID | |
| NO: 12) | |
| PM88 | tgcttagagctccaccgcggtggcggccgcttaggtatcagatggttgagctg |
| (SEQ ID | |
| NO: 13) | |
| PM89 | tgcttagagctccaccgcggtggcggccgcctattttccttccctaaaacatgac |
| (SEQ ID | |
| NO: 14) | |
| PM90 | tgcttagagctccaccgcggtggcggccgcttagaatctcaagacatgtgtggaac |
| (SEQ ID | |
| NO: 15) | |
| PM91 | tgcttagagctccaccgcggtggcggccgcttagacaggaactggtctattgca |
| (SEQ ID | |
| NO: 16) | |
| PM92 | tgcttagagctccaccgcggtggcggccgcttagacaggaactggactcaagaca |
| (SEQ ID | |
| NO: 17) | |
| PM93 | tgcttagagctccaccgcggtggcggccgcttagacaggaactggtctattgca |
| (SEQ ID | |
| NO: 18) | |
| PM94 | tgcttagagctccaccgcggtggcggccgcttaggtatcgctcggctg |
| (SEQ ID | |
| NO: 19) | |
| PM95 | tgcttagagctccaccgcggtggcggccgcttatctcaagacatgtgtggaacc |
| (SEQ ID | |
| NO: 20) | |
| PB140 | CCCCCTTTGCTTATAATTGTGTGG |
| (SEQ ID | |
| NO: 21) | |
| PB141 | ACCACCAGAACGGCCGTTAGATC |
| (SEQ ID | |
| NO: 22) | |
| PM76 | agttggatgctaacgtttct |
| (SEQ ID | |
| NO: 23) | |
| PM77 | tctatcccaacaaacagcaa |
| (SEQ ID | |
| NO: 24) | |
| ACT-F | ttctgaggttgctgctttgg |
| (SEQ ID | |
| NO: 25) | |
| ACT-R | accgacgatagatgggaagac |
| (SEQ ID | |
| NO: 26) | |

### Informal Sequence Listing:

The following provides a brief description of oligonucleotide (DNA/RNA) and peptide sequences referred to in the present disclosure. This list may not be exhaustive and other sequences may be referred to by designations known to those of skill in the art.

| Description | Sequence | SEQ ID NO: |
|---|---|---|
| Human 5- | MDKLDANVSSEEGFGSVEKVVLLTFLSTVILMAILGNLL | 1 |
| hydroxytryptamine | VMVAVCWDRQLRKIKTNYFIVSLAFADLLVSVLVMPFG | |
| Receptor 4(A)- 5- | AIELVQDIWIYGEVFCLVRTSLDVLLTTASIFHLCCISLD | |
| HTR_{4A} (Q13639-2) | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLRYTVLHRGHHQELEKLPIHNDPESLES | |
| | CF | |
| Human 5- | MDKLDANVSSEEGFGSVEKVVLLTFLSTVILMAILGNLL | 2 |
| hydroxytryptamine | VMVAVCWDRQLRKIKTNYFIVSLAFADLLVSVLVMPFG | |
| Receptor 4(B)- 5- | AIELVQDIWIYGEVFCLVRTSLDVLLTTASIFHLCCISLD | |
| HTR_{4B} (Q13639-1) | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLRDAVECGGQWESQCHPPATSPLVAAQ | |
| | PSDT | |
| Human 5- | MDKLDANVSSEEGFGSVEKVVLLTFLSTVILMAILGNLL | 3 |
| hydroxytryptamine | VMVAVCWDRQLRKIKTNYFIVSLAFADLLVSVLVMPFG | |
| Receptor 4(C)- 5- | AIELVQDIWIYGEVFCLVRTSLDVLLTTASIFHLCCISLD | |
| HTR_{4C} (Q13639-9) | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLSSGTETDRKKLWNKEEKIDQTIQMPKR | |
| | KRKKKASLSYEDLILLGRKSCFREGK | |
| Human 5- | MDKLDANVSSEEGFGSVEKVVLLTFLSTVILMAILGNLL | 4 |
| hydroxytryptamine | VMVAVCWDRQLRKIKTNYFIVSLAFADLLVSVLVMPFG | |
| Receptor 4(D)- 5- | AIELVQDIWIYGEVFCLVRTSLDVLLTTASIFHLCCISLD | |
| HTR_{4D} (Q13639-3) | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLRF | |
| Human 5- | MDKLDANVSSEEGFGSVEKVVLLTFLSTVILMAILGNLL | 5 |
| hydroxytryptamine | VMVAVCWDRQLRKIKTNYFIVSLAFADLLVSVLVMPFG | |
| Receptor 4(E)- 5- | AIELVQDIWIYGEVFCLVRTSLDVLLTTASIFHLCCISLD | |
| HTR_{4E} (X) S9 | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLSFPLLFCNRPVPV | |
| Human 5- | MDKLDANVSSEEGFGSVEKVVLLTFLSTVILMAILGNLL | 6 |
| hydroxytryptamine | VMVAVCWDRQLRKIKTNYFIVSLAFADLLVSVLVMPFG | |
| Receptor 4(F)- 5- | AIELVQDIWIYGEVFCLVRTSLDVLLTTASIFHLCCISLD | |
| HTR_{4F} (X) | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLSPVPV | |
| Human 5- | MDKLDANVSSEEGFGSVEKVVLLTFLSTVILMAILGNLL | 7 |
| hydroxytryptamine | VMVAVCWDRQLRKIKTNYFIVSLAFADLLVSVLVMPFG | |
| Receptor 4(G)- | AIELVQDIWIYGEVFCLVRTSLDVLLTTASIFHLCCISLD | |
| 5-HTR_{4G} | RYYAICCQPLVYRNKMTPLRIALMLGGCWVIPTFISFLP | |
| (Q13639-5) | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLSGCSPVSSFLLLFCNRPVPV | |
| Human 5- | MDKLDANVSSEEGFGSVEKVVLLTFLSTVILMAILGNLL | 8 |
| hydroxytryptamine | VMVAVCWDRQLRKIKTNYFIVSLAFADLLVSVLVMPFG | |
| Receptor 4(I)- | AIELVQDIWIYGEVFCLVRTSLDVLLTTASIFHLCCISLD | |
| 5-HTR_{4I} (Q13639- | RYYAICCQPLVYRNKMTPLRIALMLGGCWVIPTFISFLP | |
| 8) | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLRTDFLFDRDILARYWTKPARAGPFSGT | |
| | LSIRCLTARKPVLGDAVECGGQWESQCHPPATSPLVA | |
| | AQPSDT | |
| Human 5- | MDKLDANVSSEEGFGSVEKVVLLTFLSTVILMAILGNLL | 9 |
| hydroxytryptamine | VMVAVCWDRQLRKIKTNYFIVSLAFADLLVSVLVMPFG | |
| Receptor 4(N)- | AIELVQDIWIYGEVFCLVRTSLDVLLTTASIFHLCCISLD | |
| 5-HTR_{4N} | RYYAICCQPLVYRNKMTPLRIALMLGGCWVIPTFISFLP | |
| (Q13639-7) | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLR | |
| Embodiment of | MDKLDANVSSEEGFGSVEKVVLLTFLSTVILMAILGNLL | 10 |
| GPCR | VMVAVCWDRQLRKIKTNYFIVSLAFADLLVSVLVMPFG | |
| | AIELVQDIWIYGEVFCLVRTSLDVLLTTASIFHLCCISLD | |
| | RYYAICCQPLVYRNKMTPLRIALMLGGCWVIPTFISFLP | |
| | IMQGWNNIGIIDLIEKRKFNQNSNSTYCVFMVNKPYAIT | |
| | CSVVAFYIPFLLMVLAYYRIYVTAKEHAHQIQMLQRAG | |
| | ASSESRPQSADQHSTHRMRTETKAAKTLCIIMGCFCL | |
| | CWAPFFVTNIVDPFIDYTVPGQVWTAFLWLGYINSGLN | |
| | PFLYAFLNKSFRRAFLIILCCDDERYRRPSILGQTVPCS | |
| | TTTINGSTHVLX | |
| PM86 | tcaactatctcatatacaatctctctcgagATGGACTACAAAGACG | 11 |
| | ACGACGACAAAGGCTCAGGCgataagttggatgctaacgtttct | |
| PM87 | gcttagagctccaccgcggtggcggccgctcagaagcatgattccag | 12 |
| | gga | |
| PM88 | tgcttagagctccaccgcggtggcggccgcttaggtatcagatggtt | 13 |
| | gagctg | |
| PM89 | tgcttagagctccaccgcggtggcggccgcctattttccttccctaa | 14 |
| | aacatgac | |
| PM90 | tgcttagagctccaccgcggtggcggccgcttagaatctcaagacat | 15 |
| | gtgtggaac | |
| PM91 | tgcttagagctccaccgcggtggcggccgcttagacaggaactggt | 16 |
| | ctattgca | |
| PM92 | tgcttagagctccaccgcggtggcggccgcttagacaggaactgga | 17 |
| | ctcaagaca | |
| PM93 | tgcttagagctccaccgcggtggcggccgcttagacaggaactggt | 18 |
| | ctattgca | |
| PM94 | tgcttagagctccaccgcggtggcggccgcttaggtatcgctcggc | 19 |
| | tg | |
| PM95 | tgcttagagctccaccgcggtggcggccgcttatctcaagacatgt | 20 |
| | gtggaacc | |
| PB140 | CCCCCTTTGCTTATAATTGTGTGG | 21 |
| PB141 | ACCACCAGAACGGCCGTTAGATC | 22 |
| PM76 | agttggatgctaacgtttct | 23 |
| PM77 | tctatcccaacaaacagcaa | 24 |
| ACT-F | ttctgaggttgctgctttgg | 25 |
| ACT-R | accgacgatagatgggaagac | 26 |
| STF1 sequence | ATGAAGCTACTGTCTTCTATCGAACAAGCATGCGAT | 27 |
| | ATTTGCCGACTTAAAAAGCTCAAGTGCTCCAAAGAA | |
| | AAACCGAAGTGCGCCAAGTGTCTGAAGAACAACTG | |
| | GGAGTGTCGCTACTCTCCCAAAACCAAAAGGTCTCC | |
| | GCTGACTAGGGCACATCTGACAGAAGTGGAATCAA | |
| | GGCTAGAAAGACTGGAACAGCTATTTCTACTGATTT | |
| | TTCCTCGCGAAGACCTTGACATGATTTTGAAAATGG | |
| | ATTCTTTACAGGATATAAAAGCATTGTTAACAGGATT | |
| | ATTTGTACAAGATAATGTGAATAAAGATGCCGTCACA | |
| | GATAGATTGGCTTCAGTGGAGACTGATATGCCTCTA | |
| | ACATTGAGACAGCATAGAATAAGTGCGACATCATCA | |
| | TCGGAAGAGAGTAGTAACAAAGGTCAAAGACAGTTG | |
| | ACTGTATCTAGACCATCTAGTACAACAAAATCAGATA | |
| | ATTCGCCTCCAAAATTAGAAAGCGAGAATTTTAAGG | |
| | ATAATGAGTTGGTAACAGTAACTAATCAGCCGCTTTT | |
| | AGGCGTTGGCCTCATGGATGACGATGCGCCAGAAT | |
| | CCCCCTCTCAAATTAATGATTTTATTCCTCAGAAATT | |
| | GATTATAGAACCCAATACTCTCGAATTGAATGGTCTC | |
| | ACAGAAGAAACGCCTCATGACTTACCCAAGAATACC | |
| | GCTAAGGGCAGAGACGAAGAAGATTTTCCTCTCGAC | |
| | TATTTTCCTGTATCTGTTGAATACCCTACGGAGGAAA | |
| | ATGCGTTTGATCCGTTCCCTCCACAGGCTTTTACGC | |
| | CAGCTGCCCCTTCCATGCCTATTTCCTATGATAACG | |
| | TGAATGAAAGGGATTCTATGCCCGTTAATTCTCTTCT | |
| | TAATAGATACCCCTATCAGTTATCAGTGGCACCCAC | |
| | TTTCCCAGTGCCACCATCATCATCGAGGCAACATTT | |
| | TATGTATCCTTACGACGTTCCAGATTATGCTATTGAC | |
| | TCTGCAGCTCATCATGATAACTCCACAATTCCGTTG | |
| | GATTTTATGCCCAGGGATGCTCTTCATGGATTTGATT | |
| | GGTCTGAAGAGGATGACATGTCGGATGGCTTGCCC | |
| | TTCCTGAAAACGGACCCCAACAATAATGGGTTCTAA | |
| STF2 sequence | ATGGGTGCTCCACCTAAGAAGAAAAGAAAGGTTGCC | 28 |
| | AAAGCTTTGACTGCCAGACAACAAGAAGTCTTCGAT | |
| | TTGATTAGAGATCATATTTCTCAAACTGGTATGCCAC | |
| | CAACTAGAGCTGAAATTGCTCAAAGATTGGGTTTCA | |
| | GATCTCCAAACGCCGCTGAAGAACACTTGAAAGCTT | |
| | TGGCTAGAAAGGGTGTCATTGAAATTGTTTCTGGTG | |
| | CTTCTAGAGGTATTAGATTGTTGCAAGAAGAAGAAG | |
| | AAGGTTTGCCATTGGTTGGTAGAGTCGGTAGACCAT | |
| | CTTCTACTACTAAATCTGATAACTCTCCACCAAAGTT | |
| | GGAATCTGAAAACTTCAAAGATAACGAATTGGTTACT | |
| | GTTACAAATCAACCATTGTTAGGTGTCGGTTTGATG | |
| | GATGACGATGCTCCAGAATCTCCTTCTCAAATTAAC | |
| | GATTTCATTCCACAAAAGTTGATTATTGAACCAAACA | |
| | CTTTGGAATTGAACGGTTTGACTGAAGAAACTCCAC | |
| | ACGATTTGCCAAAGAATACTGCCAAAGGTAGAGATG | |
| | AGGAAGACTTCCCATTGGATTACTTTCCAGTTTCTGT | |
| | CGAATATCCAACTGAAGAAAACGCTTTCGATCCATTT | |
| | CCACCACAAGCCTTTACTCCAGCTGCACCATCTATG | |
| | CCAATTTCTTACGATAACGTTAATGAAAGAGATTCTA | |
| | TGCCAGTCAACTCATTGTTGAATAGATACCCATATCA | |
| | ATTGTCTGTTGCTCCAACTTTCCCAGTTCCTCCATCT | |
| | TCTTCAAGACAACACTTTATGGGTATTAACAAGGATA | |
| | TTGAGGAATGTAATGCCATCATTGAACAATTCATCGA | |
| | TTACTTGAGAACTGGTCAAGAAATGCCAATGGAAAT | |
| | GGCCGATCAAGCCATTAACGTTGTCCCAGGTATGAC | |
| | TCCAAAGACTATTTTGCACGCTGGTCCACCAATTCA | |
| | ACCAGATTGGTTGAAATCTAACGGTTTCCACGAAAT | |
| | TGAAGCTGATGTCAATGACACATCTTTGTTATTGTCT | |
| | GGTGATGCCTCTTAA | |
| STF3 sequence | ATGACTATGGATTCTGGTGCTGATAATCAACAATCTT | 29 |
| | CTTGTAAAGATTTGAAAAGATTGTTTTCTGGTACTCA | |
| | AATTTCTACTATTGCTGAATCTGAAGATTCTCAAGAA | |
| | TCTGTTGATTCTGTTACTGATTCTCAAAAAAGAAGAG | |
| | AAATTTTGTCTAGAAGACCATCTTATAGAAAAATTTT | |
| | GAATGATTTGTCTTCTATTGAACAAGCTTGTGATATT | |
| | TGTAGATTGAAAAAATTGAAATGTTCTAAAGAAAAAC | |
| | CAAAATGTGCTAAATGTTTGAAAAATAATTGGGAATG | |
| | TAGATATTCTCCAAAAACTAAAAGATCTCCATTGACT | |
| | AGAGCTCATTTGACTGAAGTTGAATCTAGATTGGAA | |
| | AGATTGGAACAATTGTTTTTGTTGATTTTTCCAAGAG | |
| | AAGATTTGGATATGATTTTGAAAATGGATTCTTTGCA | |
| | AGATATTAAAGCTTTGTTGACTGGTTTGTTTGTTCAA | |
| | GATAATGTTAATAAAGATGCTGTTACTGATAGATTGG | |
| | CTTCTGTTGAAACTGATATGCCATTGACTTTGAGACA | |
| | ACATAGAATTTCTGCTACTTCTTCTTCTGAAGAATCT | |
| | TCTAATAAAGGTCAAAGACAATTGACTGTTTCTATTG | |
| | ATTCTGCTGCTCATCATGATAATTCTACTATTCCATT | |
| | GGATTTTATGCCAAGAGATGCTTTGCATGGTTTTGAT | |
| | TGGTAA | |
| PGal4(5x): (The | CCGAGCTCTTACGCGGGTCGAAGCGGAGTACTGTC | 30 |
| underlined ATG | CTCCGAGTGGAGTACTGTCCTCCGAGCGGAGTACT | |
| is the start | GTCCTCCGAGTCGAGGGTCGAAGCGGAGTACTGTC | |
| codon) | CTCCGAGTGGAGTACTGTCCTCCGAGCGGAGTACT | |
| | GTCCTCCGAGTCGACTCTAGAGGGTATATAATG | |
| PLexA(4x): (The | CCGAGCTCTTACGCGGGTCGAAGTGCTGTATATACT | 31 |
| underlined ATG | CACAGCAAGTGGAGTACTGTCCTCCGAGAACTGTAT | |
| is the start | ATACACCCAGGGAGTCGAGGGTCGAAGTACTGTAT | |
| codon) | GAGCATACAGTAAGTGGAGTACTGTCCTCCGAGAAC | |
| | TGTATATAAATACAGTTAGTCGACTCTAGAGGGTATA | |
| | TAATG | |
| PCre | TCCTGGAAGTCTCATGGAGATTATACTTTATGCACC | 32 |
| | AGACAGTGACGTCAGCTGCCAGATCCCATGGCCGT | |
| | CATACTGTGACGTCTTTCAGACACCCCATTGACGTC | |
| | AATGGGAGAACTTTAGTATCCGTTTAGCTAGTTAGTA | |
| | CCTTTGCACGGAAATGTATTAATTAGGAGTATATTGA | |
| | GAAATAGCCGCCGACAAAAAGGAAGTCTCATAAAAG | |
| | TGTCTAACAGACAATTAGCGCAATAAGAAGAAAGAA | |
| | AACGGATTGAAGTTGAGTCGAGAATAATATGGCACC | |
| | CAGAAAACGCTTTAGGCTACTCGAATTAGGGTCACC | |
| | AATG | |
| pGal4(5x) | TCGACTCTAGAGGGTATATACCGAGCTCTTACGCGG | 33 |
| repressor | GTCGAAGCGGAGTACTGTCCTCCGAGTGGAGTACT | |
| | GTCCTCCGAGCGGAGTACTGTCCTCCGAGTCGAGG | |
| | GTCGAAGCGGAGTACTGTCCTCCGAGTGGAGTACT | |
| | GTCCTCCGAGCGGAGTACTGTCCTCCGAGGGATCC | |
| | ATG | |
| pLexA(4x) | CCGAGCTCTTACGCGGGTCGAAGTGCTGTATATACT | 34 |
| repressor | CACAGCAAGTGGAGTACTGTCCTCCGAGAACTGTAT | |
| | ATACACCCAGGGAGTCGAGGGTCGAAGTACTGTAT | |
| | GAGCATACAGTAAGTGGAGTACTGTCCTCCGAGAAC | |
| | TGTATATAAATACAGTTAGTCGACTCTAGAGGGTATA | |
| | TAATGATG | |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A biosensor comprising:
a sensing unit, the sensing unit comprising a G-protein coupled receptor (GPCR);
wherein the GPCR has a truncated and/or mutated C-terminus;
a synthetic processing unit, the synthetic processing unit comprising a signal transduction pathway comprising a synthetic transcription factor;
a synthetic response unit, the synthetic response unit comprising a recombinant signal molecule gene, where the recombinant signal molecule gene is operatively coupled to a promoter that is responsive to the processing unit,
wherein the sensing unit is in biologic communication with the processing unit and the processing unit is in biologic communication with the response unit.

2. The biosensor of claim 1, wherein the C-terminus has been truncated by 1-100 amino acids compared to a wildtype GPCR amino acid sequence, including by 1-5, 1-0, 1-20, 1-30, 1-40, 1-50, 1-60, 1-70, 1-80, 1-90 amino acids, such as 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 amino acids.

3. The biosensor of claim 1, wherein the C-terminus has been mutated to have an amino acid sequence that has 0-99% sequence identity to a wildtype GPCR amino acid sequence.

4. The biosensor of claim 1, wherein the GPCR is a GPCR isoform.

5. The biosensor of claim 1, wherein the GPCR is a serotonin receptor.

6. The biosensor of claim 1, wherein the GPCR is serotonin receptor 4 (5-HTR₄).

7. The biosensor of claim 1, wherein the GPCR is selected from the group consisting of: 5-HTR_{4A}, 5-HTR_{4B}, 5-HTR_{4C}, 5-HTR_{4D}, 5-HTR_{4E}, 5-HTR_{4F}, 5-HTR_{4G}, 5-HTR_{4I}, and 5-HTR_{4N}.

8. The biosensor of claim 1, wherein the GPCR has an amino acid sequence 85% to 100% identical to SEQ ID NO:1, 2, 3, 4, 5, 6, 7, 8, or 9.

9. The biosensor of claim 1, wherein the GPCR has the amino acid sequence SEQ ID NO: 10, where X is any amino acid(s) from 1 to 100 amino acids in length, including 1-70, 1-80, 1-90, such as 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 amino acids in length.

10. The biosensor of claim 1, further comprising an amplification unit, wherein the amplification unit is biologically coupled to the response unit.

11. The biosensor of claim 1, wherein the signal transduction pathway includes the transcription factor Ste12 or a synthetic transcription factor, where the synthetic transcription factor has a sequence about 90%-100% identical to SEQ ID NO: 27-29.

12. The biosensor of claim 11, wherein the promoter is Fig1, Fus1, or a synthetic promoter, wherein the synthetic promoter has a sequence 90% to 100% identical to SEQ ID NO: 30-32.

13. The biosensor of claim 1, wherein the recombinant signal molecule is a fluorescent protein.

14. The biosensor of claim 13, wherein the fluorescent protein is a fast maturing fluorescent protein.

15. An engineered yeast cell comprising:
a sensing unit, the sensing unit comprising:
a G-protein coupled receptor (GPCR);
a processing unit, the processing unit comprising:
a signal transduction pathway;
a response unit, the response unit comprising:
a recombinant signal molecule gene, where the recombinant signal molecule gene is operatively coupled to a promoter that is responsive to the processing unit,
wherein the sensing unit is in biologic communication with the processing
unit and the processing unit is in biologic communication with the response unit.

16. The engineered yeast cell of claim 15, wherein the yeast cell has a combination of gene deletions selected from the group consisting of: (1) a Far1 deletion; (2) a Far1 and a sst2 deletion; (3) a Far1 deletion, a sst2 deletion, and a Ste2 deletion; and (4) a Far1 deletion, a sst2 deletion, a Ste2 deletion, and a ste12 deletion.

17. A method comprising:
contacting an engineered yeast cell of claim 15 with a sample; and
detecting a signal generated by the response unit.

18. The method of claim 17, wherein the step of contacting the engineered yeast cell as in claim 11 with a sample further comprises incubating the engineered yeast cell of claim 15 with a biofuel producing cell.


SEQUENCE LISTING (34 sequences)

SEQ ID NO: 1 - protein, 387 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRYTVLHRGHHQELEKLPIHNDPESLESCF

SEQ ID NO: 2 - protein, 388 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 3 - protein, 411 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLSSGTETDRKKLWNKEEKIDQTIQMPKRKRKKKASLSYEDLILLGRKSCFREGK

SEQ ID NO: 4 - protein, 360 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRF

SEQ ID NO: 5 - protein, 371 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLSFPLLFCNRPVPV

SEQ ID NO: 6 - protein, 363 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLSPVPV

SEQ ID NO: 7 - protein, 378 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLSGCSPVSSFLLLFCNRPVPV

SEQ ID NO: 8 - protein, 428 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRTDFLFDRDILARYWTKPARAGPFSGTLSIRCLTARKPVLGDAVECGGQWESQCHPPATSPLVAAQPSDT

SEQ ID NO: 9 - protein, 359 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLR

SEQ ID NO: 10 - protein, 359 aa, synthetic construct
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLX

SEQ ID NO: 11 - DNA, 90 bp, synthetic construct
  source 1..90: mol_type = other DNA
tcaactatctcatatacaatctctctcgagatggactacaaagacgacgacgacaaaggctcaggcgataagttggatgctaacgtttct

SEQ ID NO: 12 - DNA, 50 bp, synthetic construct
  source 1..50: mol_type = other DNA
gcttagagctccaccgcggtggcggccgctcagaagcatgattccaggga

SEQ ID NO: 13 - DNA, 53 bp, synthetic construct
  source 1..53: mol_type = other DNA
tgcttagagctccaccgcggtggcggccgcttaggtatcagatggttgagctg

SEQ ID NO: 14 - DNA, 55 bp, synthetic construct
  source 1..55: mol_type = other DNA
tgcttagagctccaccgcggtggcggccgcctattttccttccctaaaacatgac

SEQ ID NO: 15 - DNA, 56 bp, synthetic construct
  source 1..56: mol_type = other DNA
tgcttagagctccaccgcggtggcggccgcttagaatctcaagacatgtgtggaac

SEQ ID NO: 16 - DNA, 54 bp, synthetic construct
  source 1..54: mol_type = other DNA
tgcttagagctccaccgcggtggcggccgcttagacaggaactggtctattgca

SEQ ID NO: 17 - DNA, 55 bp, synthetic construct
  source 1..55: mol_type = other DNA
tgcttagagctccaccgcggtggcggccgcttagacaggaactggactcaagaca

SEQ ID NO: 18 - DNA, 54 bp, synthetic construct; identical to SEQ ID NO: 16
  source 1..54: mol_type = other DNA

SEQ ID NO: 19 - DNA, 48 bp, synthetic construct
  source 1..48: mol_type = other DNA
tgcttagagctccaccgcggtggcggccgcttaggtatcgctcggctg

SEQ ID NO: 20 - DNA, 54 bp, synthetic construct
  source 1..54: mol_type = other DNA
tgcttagagctccaccgcggtggcggccgcttatctcaagacatgtgtggaacc

SEQ ID NO: 21 - DNA, 24 bp, synthetic construct
  source 1..24: mol_type = other DNA
ccccctttgcttataattgtgtgg

SEQ ID NO: 22 - DNA, 23 bp, synthetic construct
  source 1..23: mol_type = other DNA
accaccagaacggccgttagatc

SEQ ID NO: 23 - DNA, 20 bp, synthetic construct
  source 1..20: mol_type = other DNA
agttggatgctaacgtttct

SEQ ID NO: 24 - DNA, 20 bp, synthetic construct
  source 1..20: mol_type = other DNA
tctatcccaacaaacagcaa

SEQ ID NO: 25 - DNA, 20 bp, synthetic construct
  source 1..20: mol_type = other DNA
ttctgaggttgctgctttgg

SEQ ID NO: 26 - DNA, 21 bp, synthetic construct
  source 1..21: mol_type = other DNA
accgacgatagatgggaagac

SEQ ID NO: 27 - DNA, 1122 bp, synthetic construct
  source 1..1122: mol_type = other DNA
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgcgaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatctagaccatctagtacaacaaaatcagataattcgcctccaaaattagaaagcgagaattttaaggataatgagttggtaacagtaactaatcagccgcttttaggcgttggcctcatggatgacgatgcgccagaatccccctctcaaattaatgattttattcctcagaaattgattatagaacccaatactctcgaattgaatggtctcacagaagaaacgcctcatgacttacccaagaataccgctaagggcagagacgaagaagattttcctctcgactattttcctgtatctgttgaataccctacggaggaaaatgcgtttgatccgttccctccacaggcttttacgccagctgccccttccatgcctatttcctatgataacgtgaatgaaagggattctatgcccgttaattctcttcttaatagatacccctatcagttatcagtggcacccactttcccagtgccaccatcatcatcgaggcaacattttatgtatccttacgacgttccagattatgctattgactctgcagctcatcatgataactccacaattccgttggattttatgcccagggatgctcttcatggatttgattggtctgaagaggatgacatgtcggatggcttgcccttcctgaaaacggaccccaacaataatgggttctaa

SEQ ID NO: 28 - DNA, 1035 bp, synthetic construct
  source 1..1035: mol_type = other DNA
atgggtgctccacctaagaagaaaagaaaggttgccaaagctttgactgccagacaacaagaagtcttcgatttgattagagatcatatttctcaaactggtatgccaccaactagagctgaaattgctcaaagattgggtttcagatctccaaacgccgctgaagaacacttgaaagctttggctagaaagggtgtcattgaaattgtttctggtgcttctagaggtattagattgttgcaagaagaagaagaaggtttgccattggttggtagagtcggtagaccatcttctactactaaatctgataactctccaccaaagttggaatctgaaaacttcaaagataacgaattggttactgttacaaatcaaccattgttaggtgtcggtttgatggatgacgatgctccagaatctccttctcaaattaacgatttcattccacaaaagttgattattgaaccaaacactttggaattgaacggtttgactgaagaaactccacacgatttgccaaagaatactgccaaaggtagagatgaggaagacttcccattggattactttccagtttctgtcgaatatccaactgaagaaaacgctttcgatccatttccaccacaagcctttactccagctgcaccatctatgccaatttcttacgataacgttaatgaaagagattctatgccagtcaactcattgttgaatagatacccatatcaattgtctgttgctccaactttcccagttcctccatcttcttcaagacaacactttatgggtattaacaaggatattgaggaatgtaatgccatcattgaacaattcatcgattacttgagaactggtcaagaaatgccaatggaaatggccgatcaagccattaacgttgtcccaggtatgactccaaagactattttgcacgctggtccaccaattcaaccagattggttgaaatctaacggtttccacgaaattgaagctgatgtcaatgacacatctttgttattgtctggtgatgcctcttaa

SEQ ID NO: 29 - DNA, 708 bp, synthetic construct
  source 1..708: mol_type = other DNA
atgactatggattctggtgctgataatcaacaatcttcttgtaaagatttgaaaagattgttttctggtactcaaatttctactattgctgaatctgaagattctcaagaatctgttgattctgttactgattctcaaaaaagaagagaaattttgtctagaagaccatcttatagaaaaattttgaatgatttgtcttctattgaacaagcttgtgatatttgtagattgaaaaaattgaaatgttctaaagaaaaaccaaaatgtgctaaatgtttgaaaaataattgggaatgtagatattctccaaaaactaaaagatctccattgactagagctcatttgactgaagttgaatctagattggaaagattggaacaattgtttttgttgatttttccaagagaagatttggatatgattttgaaaatggattctttgcaagatattaaagctttgttgactggtttgtttgttcaagataatgttaataaagatgctgttactgatagattggcttctgttgaaactgatatgccattgactttgagacaacatagaatttctgctacttcttcttctgaagaatcttctaataaaggtcaaagacaattgactgtttctattgattctgctgctcatcatgataattctactattccattggattttatgccaagagatgctttgcatggttttgattggtaa

SEQ ID NO: 30 - DNA, 173 bp, synthetic construct
  source 1..173: mol_type = other DNA
ccgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatg

SEQ ID NO: 31 - DNA, 185 bp, synthetic construct
  source 1..185: mol_type = other DNA
ccgagctcttacgcgggtcgaagtgctgtatatactcacagcaagtggagtactgtcctccgagaactgtatatacacccagggagtcgagggtcgaagtactgtatgagcatacagtaagtggagtactgtcctccgagaactgtatataaatacagttagtcgactctagagggtatataatg

SEQ ID NO: 32 - DNA, 329 bp, synthetic construct
  source 1..329: mol_type = other DNA
tcctggaagtctcatggagattatactttatgcaccagacagtgacgtcagctgccagatcccatggccgtcatactgtgacgtctttcagacaccccattgacgtcaatgggagaactttagtatccgtttagctagttagtacctttgcacggaaatgtattaattaggagtatattgagaaatagccgccgacaaaaaggaagtctcataaaagtgtctaacagacaattagcgcaataagaagaaagaaaacggattgaagttgagtcgagaataatatggcacccagaaaacgctttaggctactcgaattagggtcaccaatg

SEQ ID NO: 33 - DNA, 179 bp, synthetic construct
  source 1..179: mol_type = other DNA
tcgactctagagggtatataccgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagggatccatg

SEQ ID NO: 34 - DNA, 188 bp, synthetic construct
  source 1..188: mol_type = other DNA
ccgagctcttacgcgggtcgaagtgctgtatatactcacagcaagtggagtactgtcctccgagaactgtatatacacccagggagtcgagggtcgaagtactgtatgagcatacagtaagtggagtactgtcctccgagaactgtatataaatacagttagtcgactctagagggtatataatgatg